(19) United States
(12) Patent Application Publication
(10) Pub. No.: US 2026/0273215 A1
(43) Pub. Date: Sep. 17, 2026

(54) Patient Interface Assembly with Thermoformable Seal

(71) Applicant: Medlands Consulting Limited, Auckland (NZ)

(72) Inventor: Alastair Edwin MCAULEY, Auckland (NZ)

(21) Appl. No.: 19/683,711

(22) Filed: May 20, 2026

Related U.S. Application Data
(63) Continuation of application No. 18/044,040, filed on Mar. 3, 2023.

(30) Foreign Application Priority Data
Sep. 18, 2020 (NZ) 768130

(51) Int. Cl. A61M 16/06; B29C 45/00; B29C 51/00; B29K 67/00; B29K 105/24; B29L 31/48

(52) U.S. Cl. CPC A61M 16/0605; A61M 16/0683; B29C 51/002; A61M 2016/0661; A61M 2205/0266; B29C 45/0001; B29K 2067/00; B29K 2105/24; B29K 2995/0025; B29K 2995/0026; B29K 2995/007; B29L 2031/4835

(57) ABSTRACT

A face shield, patient interface and methods of use thereof are described for improved respiratory therapy of patients. In particular, a face shield is disclosed that acts as a seal when used with a patient interface. The face shield may be manufactured from a low melt temperature hard thermoplastic material. The face shield may be formed initially formed to match the general contours of the face, but not customised to a specific user's face. The face shield may be crosslinked to provide shape memory to the seal and to improve its handling properties and is configured to be thermoformed to a user's face. The face shield may be customised to the patient's facial features to a second customised shape.

### TECHNICAL FIELD

Described herein are respiratory masks and related accessories. More specifically, described herein is a face shield, patient interface and related methods and uses thereof. The face shield or patient interface may be used for improving a user's breathing.

### BACKGROUND

Many people experience difficulty in sleeping because of breathing problems. These problems may result in snoring, or the more serious condition of sleep apnoea or Sleep Disordered Breathing (SDB). One treatment for Sleep Disordered Breathing involves the use of Constant Positive Airway Pressure (CPAP), bilevel or auto PAP. This involves the use of a CPAP flow generator, a breathing circuit and a CPAP mask (also known as a patient interface). The mask attaches to the patients face, covering the nose and/or mouth in order to deliver positive pressure air to the user. These devices operate to more fully open the breathing passages, thereby allowing for easier breathing.

People who suffer from SDB sleep most nights using their CPAP device and mask. CPAP masks have to be strapped to the user's head, using headgear supplied with the mask, often firmly to create a substantial seal against the users face.

The commonly available, mass manufactured CPAP masks have a flexible silicone seal that contacts the face of the user and a more rigid frame or mask base to support the cushion, connect to headgear straps and the breathing circuit. Examples of such masks are the Fisher & Paykel Healthcare (FPH) HC407 nasal mask and HC431 full face masks, variations of both masks are detailed in U.S. Pat. No. 8,479,726.

Movement throughout the night frequently leads to leaks between the mask and the face (a mask leak), these leaks can wake the user or their bed partner due to the noise generated by the escaping air or the draft that may be directed into the eyes.

Ongoing nightly use of CPAP masks, that typically have flexible silicone seals, can also cause marks and/or pressure sores on the face, commonly on the bridge of the nose. Pressure sores are often caused by commonly available mass manufactured masks are not custom made to the user's facial profile. Mask seals are also commonly called cushions, as they are typically soft and flexible, ‘cushioning’ the masks contact with the users face. They are made to approximate a generic facial profile and are made from flexible silicone, gel and/or foam in order to flex and conform to each user's facial profile. Even small amounts of flexing of the seal from interaction with the face causes pressure on the face and the small amount of pressure applied to the same location on the face over many hours throughout the night, and over many nights, can result in a skin pressure sore.

While the seal or cushion may have a relatively large contact area with the face, often a lot of the force is concentrated in the region of the face that is in contact with the thicker side wall of the seal, or other highly variable regions of the face where the seal needs to deform to match the facial contours, such as the bridge of the nose, resulting in relatively higher pressure in these locations.

Custom made masks that have less flexible seals that fit to the facial contours of the user are available, such the MetaMason mask detailed in US 2017/0080172, or the custom mask detailed in Thornton U.S. Pat. No. 6,857,428 ('428) may offer an alternative, however as they are custom made to each individual they take a lot of time clinician or technician to manufacture and are therefore very expensive, these designs are not mass manufacturable and cannot be sent directly to the user for contactless self-fitting. The '428 material and product are difficult heat and to handle in their softened state as they do not generally hold their shape, as they have not been crosslinked and therefore lack shape memory. When they are heated they are tacky and require a degree of skill from a technician to fabricate. They do not have a frame to form a handle for the user and provide support to the seal during the user fitting process. They also do not include headgear with optimal connections to the frame to minimise mask movement and leaks and they cannot be re-fitted to other individuals for reuse.

A further problem with commonly available mass manufactured CPAP masks, and in particular nasal and full-face masks, is that they tend to have a relatively large dead space, or breathing chamber, in a rigid frame. This is because the frame must contain enough space to accommodate a range of face and nose sizes, as the rigid frame cannot come into contact with the nose as the seal conforms to the face.

A further problem caused by the use of flexible silicone seals is that they do not provide stability to the mask, as they are flexible. To address this issue masks often have additional features such as forehead pads or cheek pads or rigid side arms to stabilise the mask. These tend to make the mask bulky and can create additional points for pressure sores to develop.

Many patients are also allergic to silicone that is used to manufacture CPAP mask cushions, this can result in rashes or irritation on the face where the mask contacts. There are very few masks that do not have a silicone seal that would provide users with an alternative.

The Resmed application US 2016/0271350 attempts to address the issue of cushion flexibility by inserting a thermoformable material into a silicone cushion. However, the thermoformable material is distant from the users face, there are still extensive regions of the flexible cushion that are not supported by the thermoformable material and the seal does not extend out from the frame in a low-profile manner, making it unstable. The mask still has a large conventional frame making it bulky, creating a large breathing chamber in the mask and is subject to contact with bedding causing movement and leaks. It does not disclose a hard mask seal with significantly increased contact area with the face to reduce pressure applied to the face. The seal thermoformable seal is not cross linked to improve its handling properties and it is not generally flat in cross section, or high aspect ratio, that could distribute the seal forces over a large area of the face.

US 2016/021350 does not disclose a face shield accessory that is designed to be used as with existing masks, where the face shield contacts the face and the mask and silicone seal is placed over it, significantly increasing the surface area of contact with the face to reduce pressure and prevent silicone allergies. The thermoformable material is not have a high aspect ratio so it would not be practical to form it to the face and place a CPAP mask seal over it, as the seal would not align with the thermoformable section. The relatively thick section shown would take a long time to heat up and cool down on the face. It does not teach of a mask seal or face shield that is formed into the general shape of the face and then crosslinked to impart shape memory, before being supplied to the user, to improve its handling, fitting and refitting properties.

Mask liners, such as the RemZzzs masks liners detailed in U.S. Pat. No. 8,365,733 are designed to fit between the users face and CPAP mask cushion, providing a barrier for users with silicone allergies and generally reducing irritation and assisting to create a seal. However, they are a soft, flexible fabric and as such do not reduce the amount of pressure that the silicone seal applies to the face, meaning that pressure sores can still develop.

Other mask liners or gel pads are available such the Boomerang Gel Pad from AG industries or the Resmed nose pad detailed in U.S. Pat. No. 9,999,738 provide a soft gel covering for the skin, however, again they are do not stop pressure being transmitted from the mask cushion to the face as they are flexible, not hard and rigid.

Masks are also used to ventilate patients in a number of settings, such as in hospitals or homes where ventilators are used with masks for non-invasive ventilation (NIV). These masks suffer the same issues as those used for OSA ventilation and hence reference to CPAP or other breathing devices should not be seen as limiting and the apparatus described herein may also be used in other settings that require a mask to form a seal on the face for pressurised breathing, for example in industrial and personal protection applications, such as the 3M 7000 series respirator.

It should be appreciated that it may be useful to provide a face shield or patient interface that attempts to address at least some of the above problems or at least provides the public with a choice.

Further aspects and advantages of the face shield, patient interface and methods and uses thereof will become apparent from the ensuing description that is given by way of example only.

### SUMMARY

Described herein is a face shield, patient interface and methods and uses thereof that may benefit a user's breathing. The face shield may be manufactured and shaped in a highly customisable manner. In combination with a patient interface, the face shield may provide a superior seal between the patient's face and the patient interface.

In a first aspect, there is provided a patient interface comprising:
a frame and a thermoplastic mouldable seal coupled to the frame configured to be positioned on a user's face;
wherein the seal is substantially between the frame and a user's face when the interface is positioned on the user's face; and
wherein the seal comprises cross-linked thermoplastic polymer not customised to any user's face, the cross-linked thermoplastic polymer configured to soften to be mouldable to a shape of a portion of the user's face when the seal is heated to 50 to 70° C.

The seal may be configured to conform to the contours of the user's face proximate the user's nose.

The seal cross-linked thermoplastic polymer is cross-linked to have a shape memory, the shape memory conforming to a shape of a portion of a person's face.

The seal may comprise an irradiated, cross-linked polymer.

The seal may comprise polycaprolactone.

At least a portion of the frame may be permanently attached to the seal.

The frame may define a breathing chamber in contact with pressured gas, and the seal substantially contacts the user's face in a region outside of the perimeter of the breathing chamber outlet as calculated in the coronal plane.

The frame may define a breathing chamber in contact with pressured gas, and the seal extends beyond the frame breathing chamber outlet perimeter vertically by at least 20 mm as calculated in the coronal plane.

The frame may be configured to be located at least partially superior relative to the tip of the user's nose to hold the seal away from at least a portion of the user's alar during seal moulding to the user's face.

The frame may comprise a material that provides substantial rigidity at temperatures at or below 100° C.

The frame may comprise polycarbonate.

In a second aspect, there is provided a face shield configured for use with a patient interface comprising:
inner and outer opposing surfaces, an outer edge, an inner edge and an opening in the face shield the perimeter of which is defined by the face shield inner edge;
the inner surface is configured to communicate with a patient's face or part thereof when fitted; and
the outer surface is configured to communicate with a patient interface;
and wherein the face shield is manufactured from a thermoformable polymer, the shape of the face shield on manufacture having a common first shape and the shape of the face shield after heating and moulding being a second shape customised to the patient's face.

The opening may be configured so that when a first face of the face shield is located on a patient, the opening is located about the patient's nose and/or mouth.

The inner and outer edge(s) may be at least partially curved and/or rounded.

The average thickness of the face shield, measured in a direction perpendicular to a first inner surface of the face shield and between the two opposing faces may be approximately 1 to 4 mm.

The average ratio of the distance between the inner and outer edge divided by the average thickness of the face shield may be from 5-35.

The outer surface of the face shield may be generally planar when viewed in a radial cross section.

The outer surface of the face shield may be generally concave when viewed in a radial cross section.

The common first shape may be generally flat.

The second customised shape may be contoured to follow the patient's facial contours.

The thermoformable polymer may be cross-linked.

The thermoformable polymer may have a melt temperature of 50-70° C.

The thermoformable polymer at 10-30° C. may have a hardness equal to or greater than 15 Shore D.

The thermoformable polymer at 10-30° C. may have a hardness of between 50-60 shore D.

The thermoformable polymer may be an aliphatic polyester.

The thermoformable polymer may be a polycaprolactone polymer.

At least part of the inner surface of the face shield may be configured to contact the patient's face about: a chin region, over a nasal bridge region, a cheek, an upper lip region, and combinations thereof.

The face shield may be configured to prevent contact between a patient interface and a patient's face.

In a third aspect, there is provided a patient interface for supply of gases to a patient comprising:
a face shield substantially as described above;
a frame with an inlet opening to communicate with a respirator and the frame defines a breathing chamber to communicate with the patient's nose and/or mouth;
wherein the face shield in use, is located between a patient interface and a patient's face.

The face shield may be releasably held between a patient interface and a patient's face in use.

The face shield may alternatively be fixed to a patient interface.

The face shield may prevent contact between a patient interface and a patient's face.

The face shield may form a substantially air tight connection to the patient's face and a substantially air tight connection to the patient interface.

The face shield may extend over the patient's face beyond the frame perimeter.

The frame may be manufactured from a material with a higher melt temperature than the face shield.

The frame material may have a melt temperature equal to or greater than 100° C.

The frame may have a support structure to urge engagement of the frame against at least part of the patient's face.

The frame inlet opening may be directed in an inferior direction and is located substantially posterior to a coronal plane that intersects the tip of the nose.

The frame may have between 25-50 outlet vents to vent gases from the breathing chamber.

The frame shape may be configured so that a patient can hold the frame and face shield thereon and place the face shield and a portion of the frame in water without the patient touching the water.

The frame may have a headgear connector, the headgear connector located on a midline of the frame.

The patient interface may be a nasal patient interface.

Alternatively, the patient interface may be a full-face patient interface.

In a fourth aspect, there is provided a method of customising the shape of the face shield from a common first shape to a second customised shape by the steps of:
providing a face shield substantially as described above;
heating the face shield to the material melt temperature;
placing the heated face shield onto the patient's face approximate the desired position for the face shield on the patient's face; and
letting the face shield cool and harden to the second customised shape.

The face shield material may become translucent when the melt temperature is reached.

The face shield material may become opaque when it cools to a temperature below the material melt temperature.

In a fifth aspect, there is provided the use, in the manufacture of a patient interface, of a face shield configured to be located intermediate a patient interface and a patient's face, to substantially seal the connection between a patient interface and the patient's face.

In a sixth aspect, there is provided a CPAP, APAP or BiPAP system comprising the face shield substantially as described above.

In a seventh aspect, there is provided a CPAP, APAP or BiPAP system comprising the patient interface substantially as described above.

Selected advantages of the face shield, patient interface and methods and uses thereof may include:
Provision of a fully customised seal;
The versatility to provide a face shield both as an OEM part or after market;
Minimising the need for firm or tight strapping since the seal between the patient interface and face is superior;
Minimising or preventing leakage from the patient interface particularly when the patient moves;
Minimising or preventing pressures sores since the pressure on the patient's face is even and highly customised to the patient and there are no localised pressure points;
Dead space in a patient interface frame may be minimised hence reducing frame bulk;
The patient interface is generally more stable than art solutions;
The face shield is not manufactured from silicon and provides a barrier to any silicon parts that may be present hence avoids silicon allergy issues.

### BRIEF DESCRIPTION OF THE DRAWINGS

Further aspects of the face shield, patient interface and methods and uses thereof will become apparent from the following description that is given by way of example only and with reference to the accompanying drawings in which:

FIG. 1 illustrates is a schematic diagram of a prior art CPAP blower, breathing circuit and mask in use;

FIG. 2 is a cross sectional view of a prior art nasal mask;

FIG. 3 is an illustration of a face showing some key land marks;

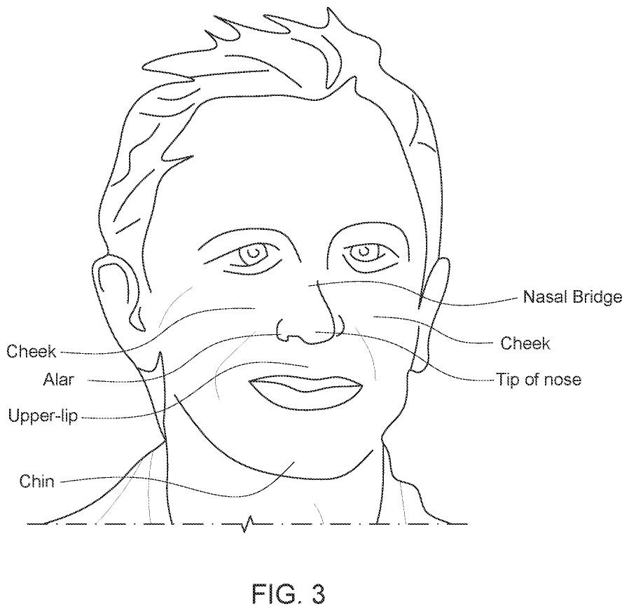

FIG. 4 is a perspective view of a full-face mask face shield in a first embodiment;

FIG. 5 is a side view of a full-face mask face shield on a user's face;

FIG. 6 is a front view of a full-face mask face shield on a user's face;

FIG. 7 is drawing showing various views of a full-face mask face shield;

FIG. 8 is a side view of the nasal mask face shield being aligned with a face;

FIG. 9 is a front view of the nasal mask face shield on a user's face;

FIG. 10 is an illustration of a combination full face and nasal mask face shield;

FIG. 11 is a schematic representation of molecule chains un-crosslinked (left) and crosslinked (right);

FIG. 12 is an illustration of an embodiment of a nasal mask with thermoformable seal;

FIG. 13 is an exploded view of the nasal mask;

FIG. 14 is a side view of the nasal mask placed of a user's face;

FIG. 15 is a side view cross section of the nasal mask placed of a user's face;

FIG. 16 is a close-up perspective view of the nasal mask seal and frame;

FIG. 17 is various views of the frame of an embodiment of patient interface;

FIG. 18 is a side view of the frame of the above embodiment;

FIG. 19 is a top view of the nasal mask on a user's face showing force vectors;

FIG. 20 is an illustration of the nasal mask placed in a bowl of hot water;

FIG. 21 is a cross sectional view of an embodiment of full-face mask;

FIG. 22 is a perspective view of a further embodiment of a nasal mask with headgear, frame and thermoformable seal;

FIG. 23 shows two detail perspective views of the embodiment of FIG. 22 nasal mask frame and seal;

FIG. 24 shows a side cross-section view of the above embodiment of mask;

FIG. 25 shows various detail views of the nasal mask frame of the above embodiment; and

FIG. 26 shows various detail views of the seal and lower frame of the above further embodiment.

### DETAILED DESCRIPTION

As noted above, described herein is a face shield, patient interface and methods and uses thereof that may benefit a user's breathing. The face shield may be manufactured and shaped in a highly customisable manner. In combination with a patient interface, the face shield may provide a superior seal between the patient's face and the patient interface.

For the purposes of this specification, the term ‘about’ or ‘approximately’ and grammatical variations thereof mean a quantity, level, degree, value, number, frequency, percentage, dimension, size, amount, weight or length that varies by as much as 30, 25, 20, 15, 10, 9, 8, 7, 6, 5, 4, 3, 2, or 1% to a reference quantity, level, degree, value, number, frequency, percentage, dimension, size, amount, weight or length.

The term ‘substantially’ or grammatical variations thereof refers to at least about 50%, for example 75%, 85%, 95% or 98%.

The term ‘comprise’ and grammatical variations thereof shall have an inclusive meaning—i.e. that it will be taken to mean an inclusion of not only the listed components it directly references, but also other non-specified components or elements.

For ease of reference, the face shield and patient interface is described below with reference to use in a CPAP system. This should not be seen as limiting since the face shield and patient interface may be used for other systems.

The term ‘face shield’ may be used interchangeably with the term ‘seal’ herein.

The term ‘patient interface’ may be used interchangeably with the term ‘mask’ herein.

The terms ‘patient’ may be used interchangeably with the term ‘user’ herein.

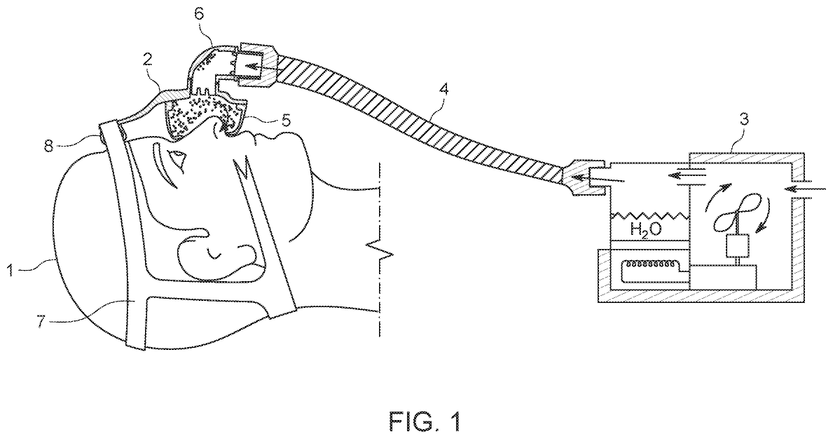

FIG. 1 is a schematic diagram of a Continuous Positive Airway Pressure (CPAP) system where patient 1 is receiving humidified pressurised gases through interface 2. The interface is connected to the CPAP flow generator 3 via breathing circuit 4. It should be understood that the flow generator 3 could also be an APAP (Auto Positive Airway Pressure), a BiPAP (Bi-level Positive Airway Pressure) or numerous other forms of respiratory therapy.

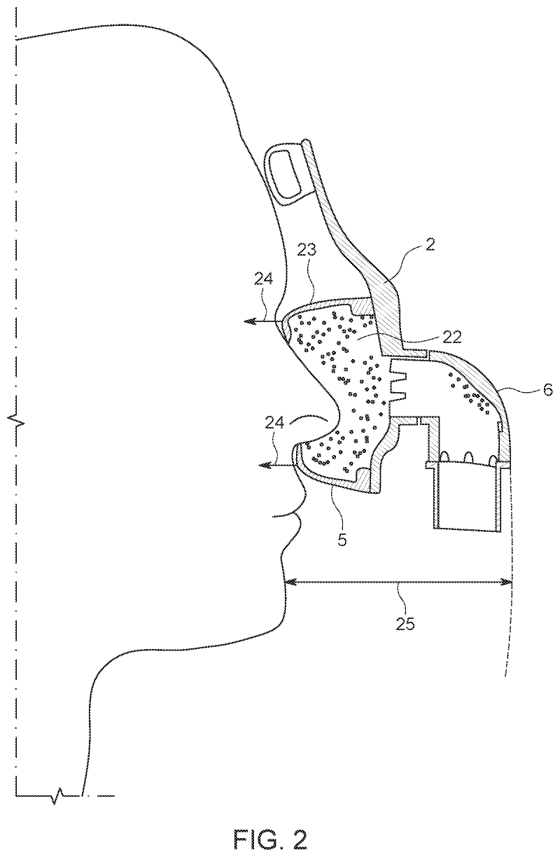

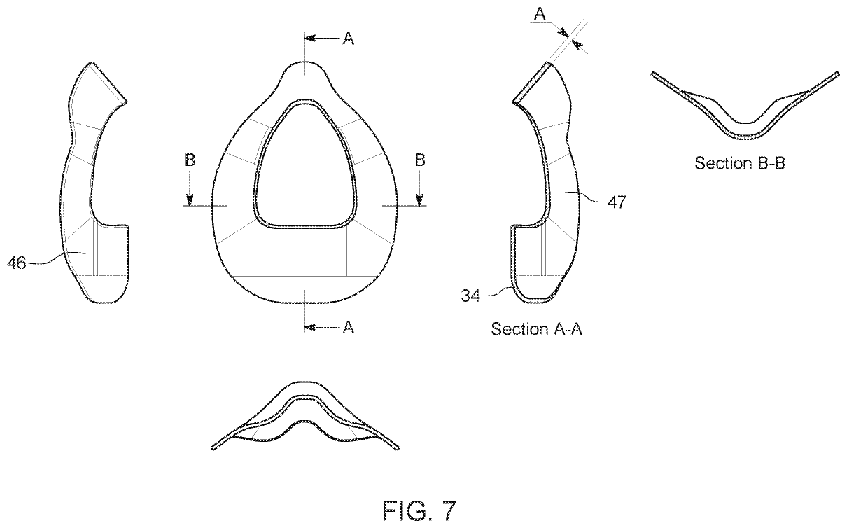

FIGS. 1-2 show a prior art nasal CPAP mask, or patient interface, in cross section. Patient interface 2 is a general representation of the form of the Fisher & Paykel Healthcare (FPH) HC407 or Zest nasal masks, as shown in U.S. Pat. No. 8,479,726 FIG. 7. Interface 2 is shown in cross section and has a flexible seal 5 to create a substantial air seal, or substantial air tight connection, against the users face. The seals are made from silicone, gel and/or foam with a hardness of Shore A 5-50, with the average silicone seal being Shore A 40. In addition to being made from soft materials they are also very thin, having face contacting wall sections of 0.2-1.4 mm and side wall sections of 2-4 mm and are designed to flex where they contact the face, with large unsupported regions where the seal is directed inwards against the surface of the face.

It has a rigid frame shown as being cross hatched that supports the seal, an elbow 6 to connect the circuit 4 to the interface, headgear 7 to secure the interface to the patients head and a forehead support 8 to stabilise the interface. The headgear 7 has four points of connection to the frame, however the lower strap of the headgear is shown as being truncated to illustrate other details of the interface.

The flow generator supplies air at positive pressure that flows generally in the direction of the arrows to the patient as well as from the patient to be vented out the bias vent holes shown in the elbow as dots.

It should be noted that the terms ‘interface’, ‘mask’ and ‘mask assembly’ can be used interchangeably and mean the same thing. The terms ‘mask seal’, ‘seal’ and ‘cushion’ when used to describe mask components can be used interchangeably and mean the same thing. Mask ‘base’ is also known as mask ‘frame’ and mask ‘body’. A ‘CPAP mask’ is also equivalent to a ‘respiratory mask’ or may simply per referred to as a ‘mask’. The term ‘air seal’ refers to the action or verb of preventing air or gas from escaping and shall mean the same as the term ‘air connection’ and shall not be confused with the mask component or noun use of the term ‘mask seal’ or ‘seal’.

It should be noted that the description of the geometry, surfaces, sizing and distances described in this specification that related to the thermoformable seal and face shield, unless otherwise stated, relate to their initial mass manufactured form, before they are thermoformed to the users face. Where the face shield or thermoformable mask seal is discussed as forming a substantial air seal with the user's face this occurs after the face shield or mask seal has been thermoformed to the user's face.

All human anatomical references to directions and planes for mask and face shield components are made with reference to the mask or face shield being in use on a user's head and relate to the users head as a reference. The ‘user’ 1 may be a ‘patient’ 1 or a ‘mask user’, for example the latter would apply to an industrial and/or healthcare worker applications of the present invention, where the user may not be considered to be a patient.

It should be noted that all figures in the specification that show the face shield and thermoformable seals from the mask assemblies show these components in their initial mass manufactured form. The figures that show these components on a user's face are not shown in a form that has been thermoformed to the individual users face, as they do not match the exact contours of the image of the head in these figures. It should be understood that in use these components will be thermoformed to the individual users face and will be in close contact with the users face in order to substantially create an air seal between these components and the users face.

Temperature references are made with reference to standard atmospheric conditions, such as pressure, at sea level.

### Thermoformable Full Face Mask Face Shield

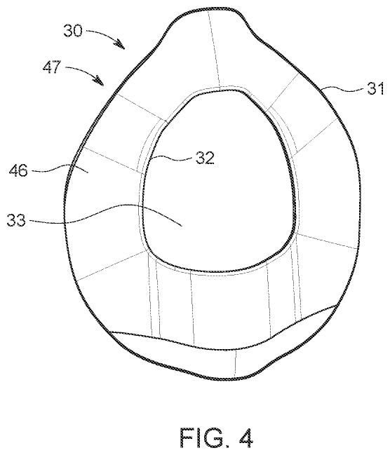

In a first embodiment, there is provided a patient interface as shown in FIGS. 4-7 being a thermoformable full face mask face shield 30 that is an accessory to be used with other readily available full-face CPAP masks that have generally soft silicone seals. The face shield has an outer edge 31 and an inner edge 32 that defines an opening 33. The inner and/or outer edges may be partially, or full, curved or rounded to improve the comfort on the face.

The face shield 30 has an outer surface 46 that comes into contact with a CPAP mask seal and an inner surface 47 that contacts the user's face. The inner surface and outer surface are defined by three regions, a nasal bridge region a chin region and a cheek region that is located between the nasal bridge and chin regions. The full-face shield has a generally rounded triangular form and is shaped to match the general contours of the user's 1 face in the nasal bridge region 35, cheek region 36 and chin region 37, but is not initially customised to a particular user's face. Inner surface 47 has a concave form in the nasal bridge region, when viewed through the transverse plane. Alternatively, the face shield may be supplied generally flat and then formed to the contours of the face during fitting.

The face shield 30 may be formed from a low melt temperature, hard thermoplastic material, for example, into the shape shown in FIG. 4, using injection moulding. The preferred material is polycaprolactone and is described further below.

It is also possible to form the face shield by injection moulding and then cutting, for example to form different sizes. They can be formed from flat sheet using vacuum forming and cutting from sheet stock, or simply cutting from flat sheet stock. If sheet stock is used, the sheet may be crosslinked before or after cutting. If formed using vacuum forming it may be useful to crosslink the sheet before vacuum forming to improve the handling properties of the sheet.

After being formed into an initial shape, the face shield material is then crosslinked. Crosslinking improves the handling of the face shield while it is in a softened state for thermoforming to the users face, making it less sticky, holding it generally in its pre-softened shape. Crosslinking can be achieved through the use of irradiation and these details and benefits are further described below.

The face shield is heated to between 50-70° C., or to above 60° C., for example by placing it in hot water in order to transition the face shield into its softened, thermoformable state. The face shield turns clear or translucent when it has reached its melt temperature, providing a visual indication that it is ready to be moulded to the shape of the users face. It is then placed on the users face, to form to the contours of the face. The user may need to press the face shield lightly against the face, lie on their back and/or place their CPAP mask over the face shield temporarily to form the face shield to the contours of the face. It then sets on the face as it cools below its melt temperature, thus fitting the face shield to the users face. Providing the face shield with preformed contours of the face (rather than a flat sheet) makes this process easier for the user, as it more closely matched the facial contours before fitting and is less likely to form creases. Alternatively, is it possible to form the face shield from a flat sheet, lowering the cost of manufacture and making it more convenient to ship.

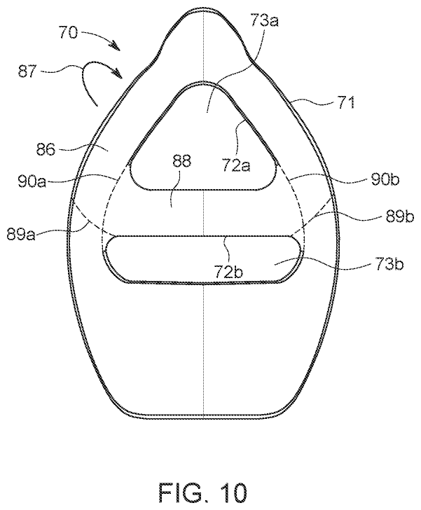

Information such as branding, sizing, company and/or product names may be printed on the inner and/or outer surface, or any surface of the thermoformable seal/face shield. This printing may be the same colour as the cooled device, for example white, so it will not appear in the cooled state, but will appear when the device is heated above its melt temperature as the device turns clear or translucent, revealing the printing, this will highlight the technology and brand similar a water mark. The word ‘Ready’ or symbols such as a tick may be used to further indicate to the user that the product is ready to be fit to the face or trimmed, as they will only appear once the seal is heated above its melt temperature. The printing could also be used to reveal where to cut the product, to change size from large to medium or small or to change product as shown in FIG. 10. That way the markings would only appear when it is in the softened state and not detract from the product while in use. The product may be resized, to a smaller size or different style of mask, for example by cutting with house hold scissors, while in the softened or heated state.

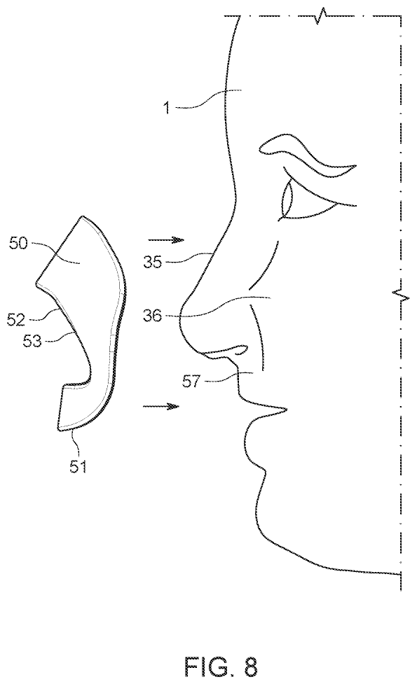

The fitted or customised full-face shield 30 is placed on the face and then a full-face CPAP mask, that communicates with the nose and mouth, is placed on top of the face shield. The user's face, face shield and CPAP mask are releasably in contact with each other and the face shield is held in place during use of the CPAP mask by the force the CPAP mask and its headgear apply to the face, in the same manner as shown in FIG. 8 of U.S. Pat. No. 8,365,733 that shows a fabric mask liner between the face and a full-face CPAP mask. In use opening 33 allows for communication of the gas delivered from the CPAP with the patient's airways, via the nose and/or mouth.

The inner surface 47 of the face shield is configured to be in non-adhering communication with the users face, creating a substantial air seal, or air tight connection, with the face. The outer surface 46 is in non-adhering communication with the seal of the CPAP mask, creating a substantial air seal, or air connection, with the seal of a CPAP mask. Non-adhering means the components are not glued, bonded or stuck together using any form of adhesives or chemicals.

In a variation, the CPAP mask seal may be glued or bonded to the face shield outer surface, adhering the mask seal to the face shield, for example, using an adhesive that will bond with the silicone seal. This will further improve the air seal between the mask and the face shield.

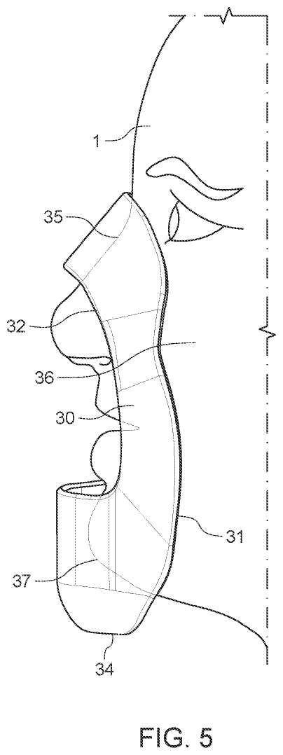

FIG. 5 shows a side view of the full-face shield 30 on a patient's 1 face. The inner surface 47 may have a generally concave region forming a chin support 34 that cups the users chin 37. The chin support 34 fitting under the chin allows the face shield to support the users lower jaw or mandible, preventing the mandible from lowering which can cause a poor seal and/or unwanted mouth leakage. Many patients use chin straps to reduce this mandible movement and the face shield provides the same function by contouring under the chin. This chin support can benefit both users of full-face masks, nasal masks and nasal pillow masks. For example, a nasal mask user could use the face shield 70 shown in FIG. 10 that prevents contact of the nasal seal with the face and can contour under the chin, assisting to keep the mouth closed.

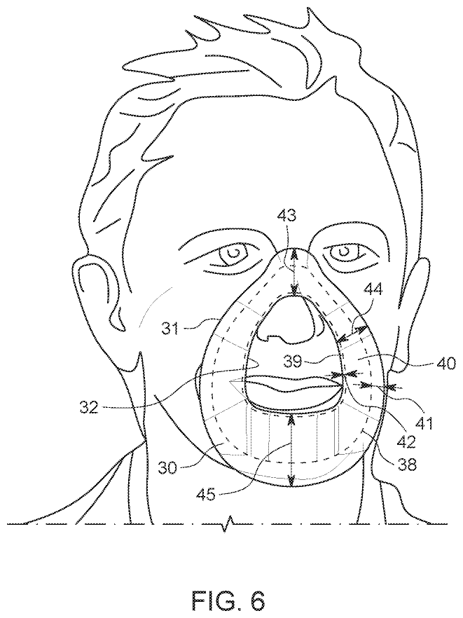

FIG. 6 shows a front view of the full-face shield on a patient's 1 face. The outer perimeter of full-face seal contact 38 and inner perimeter of full-face seal contact 39 are indicated by the dashed lines. A typical full-face seal will contact the full-face shield 30 in the seal face contact area 40 between the outer 38 and inner 39 face seal contact perimeters. For a medium full-face seal this area is typically about 50 cm². The area of the face shield outer surface 46 and inner surface 47 shall be 80 cm²-120 cm² or 60-140% larger than the facial contact area 40 of the mask seal, in order to reduce the average pressure applied to the face, by a factor of 1.6-2.4. The face shield outer edge 31 extends, indicated by arrow 41, beyond the outer perimeter of seal contact 38 in order to ensure that the seal does not contact the face. The perimeter of outer edge 31 is larger than the outer perimeter of seal contact 38. If in use the mask seal does extend beyond the face shield outer edge 31, and contacts the face, this could lead to pressure sores, leaks and skin irritation. The outer edge may extend in the range of 2 mm-20 mm, per side, beyond the outer edge of the interface seal facial contact area 40.

The inner edge 32 extends inward, as indicated by arrows 42, beyond the inner perimeter of the seal contact 39, in order to substantially prevent the interface seal from contacting the face. The perimeter of inner edge 32 shall be less than, or equal to, the inner perimeter of seal contact 39. The inner edge of interface seal generally applies less pressure to the face than the outer edge and the face shield is less sensitive to the issues caused by the inner edge contacting the face through opening 33, therefore, less inner extension is required. For example, the inner edge could be sized to approximately match that of the inner edge of the interface seal or it could extend up to 10 mm per side in from the inner perimeter of the seal contact 39. It should be noted that as the interface seal is applied to the face it can becomes wider, so an initial spacing between the outer edge of 20 mm, while not in use, could become 2 mm while in use.

FIG. 7 shows a medium sized full-face shield 30 in various plan, elevation, cross section and side views, with dimensions in mm. The face shield may be on average 1.0 mm-4.0 mm thick or, on average 2.0 mm-3.0 mm thick in a direction that is perpendicular to the inner surface 47, at each point of measurement. FIG. 7 shows a radial cross section A-A with a thickness of 2.5 mm. Radial cross section, in this specification, shall mean a cross sectional cut that is perpendicular to the outer edge of the face shield or seal as viewed in the coronal plane.

The outer surface 46 of the face shield may be generally flat in radial cross section, for example, in the cheek region shown by radial cross section B-B of FIG. 7, or it may be generally concave as viewed in the same cross section, excluding the chin support region 34. A concave surface may create a better mating and sealing surface with the seal of the CPAP mask, and the concave surface may also provide more rigidity to the structure of the face shield. The concave surface may be created by varying wall section, leaving inner surface 47 generally planar to the surfaces of the face, or the inner surface may be slightly convex, excluding the chin support region. Alternatively, outer surface 46 may only be concave in the cheek region, while the nasal bridge region may be generally flat and the chin region may be convex or flat.

The following dimensions relate to the overall widths and heights of a medium sized full-face shield that is sized to fit a medium size full face CPAP mask. The full-face shield should be 95-115 mm wide (lateral direction), or 100-110 mm wide. It should be 130-150 mm high (superior/inferior direction) for a version with a chin support 34. A version without a chin support should be 110-125 mm in height. Opening 33 should between 65-75 mm in height and 50-60 mm wide. The radial distance 43 and 44 from the inner edge 32 to the outer edge 31 in the nasal bridge region and cheek region should be between 20-35 mm, measured in the general plane, or major axis, of the outer surface 46 at each location. The distance 45 in the cheek region should be between 20-35 mm for a face shield without a chin support 34 or between 35-55 mm for a face shield with a chin support, as measured in the coronal plane.

The small sized full-face shield will have similar width dimensions to the medium size but be 5-15 mm less in height. The large sized full-face shield will also have similar width dimensions to the medium size but be 5-15 mm more in height. The change in height shall apply to both the outer height and the height of opening 33.

FIGS. 6-7 show the thermoformable face shield 30 extending out from opening 33 in a generally planar manner (as shown in radial cross section) creating a shield with an aspect ratio of between 5-35, or between 6.67-17.5 (ratio=distance between inner edge 32 and outer edge 31 divided by the average thickness), in the nasal bridge and/or cheek regions. This high aspect ratio creates a thermoformable hard face shield that has increased contact area, relative to the CPAP mask seal, to reduce pressure on the face while being thin in the other direction (2-3 mm) makes it practical to heat and thermoform. This high aspect ratio also provides a generally planar, or slightly concave, outer surface 46 for the CPAP mask seal to mate against in to create a substantial air seal, or air connection, between the two components and the face. If the face shield was significantly thicker it would be impractical to heat and thermoform to the users face as it would take a long time to heat and cool. Increased cooling time can lead to the user inadvertently moving the seal during the cooling phase leading to unwanted seal deformation and a poor fit to the face. Having a substantially concave outer surface would, or one that was narrower than the seal face contacting area 40, would also make it difficult to align the CPAP mask seal with the face shield, reducing the contact area between the two leading to a poor air seal.

The thermoplastic should be relatively hard (and hence rigid) while in use at room temperature or up to body temperature. The face shield should be hard in order to distribute the forces that the mask and seal apply to the face over a large area to reduce the pressure applied to the face. The harness should be at least 15 Shore D (ASTM D 2240 55) or 50-60 Shore D.

The face shield can be used as an accessory with currently available CPAP masks and prevents or reduces contact between the silicone seal and the users face reducing silicone skin irritation and pressure sores. The face shield provides a rigid shield, or barrier, between the face and the CPAP mask that is placed over the face shield. The face shield covers the face in a similar manner to that of a traditional fabric mask liner, however as it is thicker and more rigid it more evenly distributes the CPAP mask or interface sealing forces over a larger area, reducing the pressure applied to the face from the interface. Gel pads are thicker however, they are also very flexible and do not distribute the seal forces over as large an area as the hard face shield. The face shield also allows the CPAP mask to be tightened more if necessary, increasing the masks seal contact area and improving the seal without causing discomfort as the force is distributed over the larger contact area of the face shield. These advantages are further described below.

The contact area between the face and the face shield is 60-140% larger than the typical contact area between a mask cushion and the face and the increased area provides improved sealing performance. This increased contact area can improve the sealing performance over difficult regions of the face, such as the bridge of the nose or areas that may be creased due to age or covered by facial hair such as users with moustaches.

Furthermore, as the face shield does not bond or stick to the face in the manner that gel pad does, it allows small amounts of air to pass between the face and the face shield throughout the night, allowing the skin to breath, reducing irritation and sweating. It should be noted that this small amount of leakage, that may be in the order of 5 litres per minute at 10 cm H20 pressure will not interrupt therapy and is much smaller than would be considered an undesirable mask leak, this is still considered to be a substantial air seal or air connection.

The face shield is available in a range of sizes, for example, small, medium and large, corresponding to small, medium and large masks. It is also available in a range of mask styles such as for full face masks, nasal masks, nasal pillow masks, hybrid nasal-full face masks and total face masks that additionally contact and seal above the eyes. The face shield may also be used in combination with anaesthesia masks or where gasses have to be applied to the patient during an operation. This may be of particular benefit where the patient as a facial injury, burn or sensitivity that may be aggravated by direct contact with the mask, or a facial deformity that a conventional mask may have difficulty matching and sealing on.

The inner and/or outer surfaces of the face shield or thermoformable seal may be smooth or have a frosted texture. The frosted texture may provide a more comfortable surface for skin contact and a surface that is less sticky to the silicone seal reducing creases in the seal. The inner and outer surfaces may additionally have one or more groves or protrusions running generally parallel to the outer or inner edge, in a concentric manner. These grooves or protrusions may be 0.5-2 mm deep or high and run partially or entirely around opening 33 or breathing chamber of mask. The grooves or protrusions may be in the form of a half round, square or rounded triangular shape. The benefit of the grooves or protrusions is that they may improve the sealing performance between the outer surface and the mask seal and/or the inner surface and the face by creating localised points of increased contact in a similar principal to tread on a car tyre. These grooves and/or protrusions may not be located on the inner surface in the nasal bridge region and the skin is thinner there and may not tolerate such features, where as other areas such as the cheek or chin would tolerate such features. The groove and protrusion features only apply to the inner, face contacting region of the thermoformable mask seal.

The face shield may have its own headgear strap, or straps, similar to that of a CPAP mask in order to be self-secured to the users face. The straps may also assist the face shield to act as a chin support, reducing mandible movement and mouth leak.

### Thermoformable Nasal Mask Face Shield

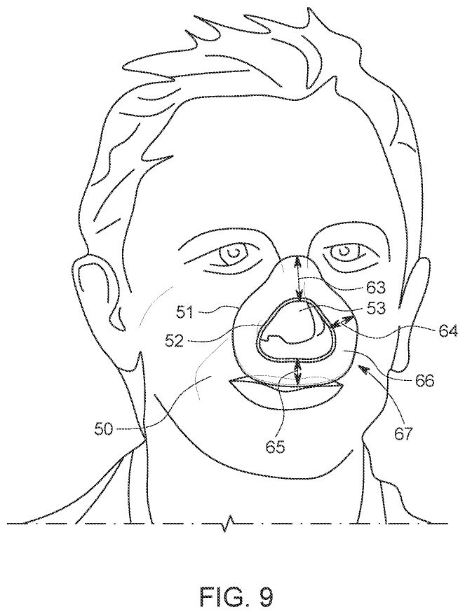

In a second embodiment, there is provided a patient interface is shown in FIGS. 8-9 as a thermoformable nasal mask face shield 50 that is an accessory to be used with other readily available nasal CPAP masks that have soft seals. The nasal face shield has an outer edge 51 and an inner edge 52 that defines an opening 53. The face shield 50 has an outer surface 66 that comes into contact with a CPAP mask seal and an inner surface 67 that contacts the users 1 face. The nasal face shield is shaped to match the general contours of the users 1 face in the nasal bridge region 35, cheek region 36 and upper lip region 57 where it contacts the face. Inner surface 67 has a concave form in the nasal bridge region, when viewed through the transverse plane. Alternatively, it may be supplied generally flat and then formed to the contours of the face during fitting.

It will be appreciated, by someone skilled in the art, that the general description of the full-face shield design, function and benefits also apply to the nasal mask face shield.

The following dimensions relate to the overall widths and heights of a medium sized nasal face shield that is sized to fit a medium nasal CPAP mask. The nasal shield should be 60-80 mm wide (lateral direction) or 65-75 mm wide. It should be 65-85 mm high (superior/inferior direction) or 70-80 mm wide. Opening 53 should between 30-50 mm in height and 30-50 mm wide. The distances 63, 64, 65 from the inner edge 52 to the outer edge 51 in the nasal bridge 35, cheek 36 and upper lip 57 regions should be between 15-30 mm, measured in the plane of the outer surface 66 at each location.

The small sized nasal mask shield will have similar width dimensions to the medium size but be 5-15 mm less in height. The large sized nasal face shield will also have similar width dimensions to the medium size but be 5-15 mm more in height. Again, height changed apply to the external height and the height of opening 53.

### Thermoformable Combination Full Face and Nasal Mask Face Shield

FIG. 10 shows a third embodiment of the present invention, a thermoformable combination full face and nasal mask face shield 70 that can be used as an accessory with either a full-face mask or a nasal mask. It will be appreciated, by someone skilled in the art, that the general description of the first and second embodiments design, function and benefits also apply to the combination full face mask and nasal mask face shield.

Face shield 70 has a nose opening 73a and a mouth opening 73b, defined by two inner edges, nasal inner edge 72a and mouth inner edge 72b. The two openings allowing communication between the CPAP mask and patients airways via the nose and mouth. Face shield 70 has upper lip cross member 88 that covers the upper lip 57 thus shielding contact between the upper lip and the lower section of a nasal mask seal. Face shield 70 can also contour under the users chin in the same manner as detailed in the first embodiment. Thus, it may assist in stabilising the mandible or jaw, keeping it closed for nasal mask and/or full-face mask users, preventing the need for a chin strap.

If nasal mask users do not want the under-chin support region they can cut face shield 70 through region indicated by the doted lines (89a and 89b), thus creating a nasal mask face shield similar to that shown the second embodiment. The doted lines 89a and 89b may be marketed on the outer or inner surface of the face shield, for example, by laser marking, printing to formed during injection moulding, in order to indicate, to the users, the region to cut. Alternatively, if a full-face mask user did not want cross member 88 they could cut through the region indicated by the doted lines 90a and 90b, thus creating a full-face shield similar to that shown in the first embodiment. This may be useful design as it would reduce the variations or SKU's that need to be tooled, manufactured and distributed. Doted lines 90a and 90b may also be marketed on the face shield in the same manner as described for doted lines 89a and 89b.

Another variation of the face shield 70 may have no opening 73b. This would serve to cover the mouth preventing or reducing air leakages from the mouth while the user was wearing a nasal mask or nasal pillow mask.

### Thermoformable Nasal Pillow Mask Face Shield

In a fourth embodiment, there is provided a patient interface comprising a thermoformable nasal pillow mask face shield that is an accessory to be used with other readily available nasal pillow CPAP masks that have silicone, gel, foam or generally soft seals. The nasal pillow mask face shield would contact the nose around the user's nares to prevent contact between the silicone seal of the nasal pillow mask and the user's nose. It will be appreciated, by someone skilled in the art, that the general description of the other face shield embodiments design, function and benefits also apply to the combination nasal pillow mask face shield.

The nasal pillow mask face shield may additional contact the upper lip, in a similar manner to the nasal mask face shield, and/or the tip of the nose to further reduce contact with the mask and stabilise the face shield. In addition, it may also contact the cheek region to provide further support.

The nasal pillow face shield may have two openings that allow each individual pillow of the nasal pillow mask to communicate with each nares. Alternatively, it may have one opening that communicated with both nares, for masks that primarily contact the nose around the nares, do not contact the nasal bridge region, and have only one outlet from their seal, such as the FPH Evora nasal mask.

### Thermoformable Nasal Bridge Face Shield

In a fifth embodiment, there is provided a patient interface comprising thermoformable nasal bridge face shield that is an accessory to be used with other readily available full face or nasal CPAP masks that have silicone, gel, foam or generally soft seals. The nasal bridge face shield contacts the nasal bridge and cheek region of the users face, in the same manner as the gel pad shown in U.S. Pat. No. 9,999,738 FIG. 4. It will be appreciated, by someone skilled in the art, that the general description of the other face shield embodiments design, function and benefits also apply to the nasal bridge face shield.

The nasal bridge face shield is generally in the form of an inverted letter “V”. The lower (inferior) and lateral edges of the nasal bridge shield may be tapered down to be thinner than the general thickness of other regions. The tapered region may end at a point or have a small radius. A point 1 mm from the edge should be no thicker than 1 mm. This tapering corresponds to the region where the CPAP mask seal passed from the nasal bridge seal to the users face. The benefit if this tapered region is to allow the CPAP mask seal to transition from the outer surface of the face shield to the users face without encountering an abrupt step that may air leaking from this region. The taper can be on the inner surface, outer surface or both surfaces. The tapered region will be at least 2 mm long and at least 5 mm long.

### Polycaprolactone—Low Melt Temperature Hard Thermoplastic Material

The face shield or CPAP mask/interface seal may be constructed from Polycaprolactone (PCL) polymer or by using another aliphatic polyester. One or more of the polycaprolactone polymers may have the formula:

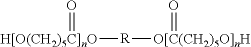

Where R is an aliphatic hydrocarbon.

TONE polycaprolactone polymers are described in U.S. Pat. Nos. 4,784,123 and 5,112,225 and product literature of Union Carbide Corporation, all incorporated here by reference, as including homopolymers, block copolymers graft copolymers, or other polymers containing epsilon-caprolactone. Polymerization may be initiated using a diol, for example and without limitation, ethylene glycol, diethylene glycol, neopentyl glycol, butane diol, hexane diol, or any other appropriate diol.

PCL is also known as Poly (hexano-6-lactone), 2-Oxepanone homopolymer and also 6-Caprolactone polymer and has a density of 1.14-1.15 g/cm³.

Another example of a suitable PCL polymer is CAPA 6500, a thermoplastic linear polyester derived from caprolactone monomer. CAPA 6500 is supplied by Perstorp with a mean molecular weight of 50,000 and a melting point of 58-60° C.

Polycaprolactone is a relatively hard plastic, with a Shore D hardness of 55, or between 50-60. As calculated at 25° C. or as stipulated in ASTM D 2240 55. This makes it very tough, for example, like nylon, it but softens to a putty-like consistency when heated above 58-60° C., for example by placing it in hot water. This low melt temperature enables it to be handled and placed on the face without burning the skin or causing discomfort. Polycaprolactone also has a relatively low heat capacity, relative to water, which further reduced the amount of energy that can be transferred to the skin during the thermoforming process. Furthermore, as it has a relatively low rate of thermal conductivity any energy or heat transfer to the skin is slow.

As polycaprolactone is hard and rigid when cooled, at room or skin temperature, relative to commonly used low temperature human body mouldable materials like EVA, therefore less volume of PCL is required to achieve the desired mechanical properties of the product, such as flex or strength. This lower volume further improves the handling properties by reducing the amount of energy in the heated product, reducing heating and cooling times and reducing the amount of energy that could be transferred to the user's skin during fitting.

The polycaprolactone has a melting point of 58-60° C. This enables the face shield or seal to be softened, for example by placing it in hot water, and comfortably placed on the face without burning the face and not become soft during use due to contact with the face that may be about 37° C.

Most relatively rigid thermoplastics have a melting temperature between 100-300° C. and would burn the face if used in this manner. Other thermoplastics are available that have a melt temperature below 100° C., such as Ethylene-vinyl acetate (EVA) that is commonly used is mouldable sports mouth guards. However, EVA still has a higher melt temperature of between 90-120° C. which is higher about 50% than the melt temperature of PCL. The melt temperature can be lowered with different EVA blends or with the addition of plasticisers however this lowers the hardness further below desired levels, and plasticisers are often not biocompatible. EVA is relatively flexible and would not have the required rigidity to provide the desired level of support to prevent localised cushion forces acting on the face, unless they were very thick that would result in a product that was too bulky to practically use.

The PCL can also be blended with other materials to create a blended materials, or copolymers, for use in forming the seal or face shield with a different feel on the skin. Any suitable blend should have a melt temperature between 50-70° C. and a hardness of more than 15 Shore D or a hardness between 50-60 Shore D.

### Crosslinking—Irradiation and Shape Memory

Crosslinking the face shield, or mask seal, imparts shape memory into the part. This means when the face shield is heating above its melting temperature of 60° C. it becomes soft but still holds its general shape. Without crosslinking it is very difficult to handle the melted material as it would tend to flow like a thick liquid and not hold its general form. Crosslinking allows the material to be in a softened state, above its melt temperature, while still retaining its general form, so it can be placed against the face and formed to the shape of the face.

The crosslinked polycaprolactone is then allowed to cool below its melt temperature of 60° C. This can take between 1-4 minutes, while the part is in contact with the face, thus setting into the form of the user's facial profile and hence being customised to the user. Crosslinking will also provide shape memory allowing the face shield to return to approximately is original injection moulded form after being reheated above its melt temperature, allowing it to be heated and refit to the same or a different users, a large number of times, without degradation of its form or mechanical properties.

After being injection moulded into its initial non-customised form the face shield or CPAP mask/interface seal undergoes irradiation to crosslink the polymer chains. Crosslinking can be achieved through the use of gamma radiation or electron beam (E-Beam) radiation or any other known methods. E-Beam crosslinking allows for more precision and uniformity of the absorbed radiation does.

Crosslinking is the interconnection of adjacent long molecules with three-dimensional networks of bonds (crosslinking) induced by chemical treatment or electron-beam (E Beam) treatment. Electron-beam processing of thermoplastic material results in an array of enhancements, such as an increase in tensile strength and resistance to abrasions, stress cracking and imparts three-dimensional shape memory into the product. E Beam treatment rather than chemical treatment allows the product to be crosslinked in its final moulded form, for example the seal and frame that have been over moulded to each other, without adversely affecting the properties of the frame, that does not need to be crosslinked, but must be subjected to the crosslinking process as it is bonded to the seal.

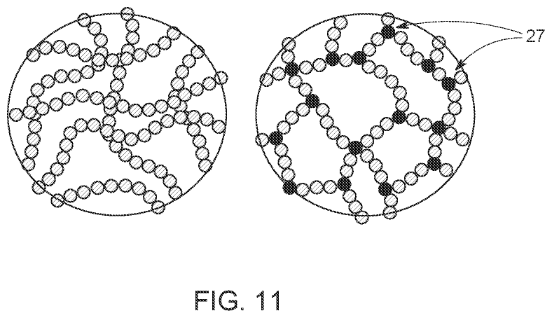

FIG. 11 shows individual polymer chains on the left and polymer chains that have been boned or crosslinked 27 on the right. The bonding or crosslinking locks the individual chains together in a three-dimensional network imparting shape memory into the material. Although FIG. 11 schematically shows this in two dimensions, it is understood that this happens in three dimensions. Without this crosslinking, when the material is heated to above its melt temperature the polymer chains are free to move in an unconstrained manner. This is essentially the point at which the intermolecular bonds are no longer strong enough to hold together and often defines the transition from a solid to a molten or liquid state.

It should also be understood that this crosslinking of the injection moulded or otherwise formed component is very different from the crosslinking of raw plastic material for injection moulding. Crosslinking of raw materials does not produce three dimensional components that have shape memory as the material was not crosslinked in its final form.

The crosslinking of polymer chains through electron-beam processing can change a thermoplastic material into a thermoset. When polymers are crosslinked, the molecular movement is severely impeded, making the polymer stable against heat. This locking together of molecules is the origin of all of the benefits of crosslinking, including imparting three-dimensional shape memory.

Conventional thermoset plastics or elastomers cannot be melted and re-shaped after they are cured. However, crosslinking polycaprolactone to the desired level of the present invention results in a material that has properties of both a thermoplastic and a thermoset material, or somewhere between the two. It can be heated above its thermoplastic melt temperature of 58-60° C., but in this semi-molten state it retains its three-dimensional form. It behaves somewhat like a very soft rubber material, or putty, for example with hardness of less than 10 Shore A, which is significantly less that the hardness of commonly available CPAP mask silicone seals that are typically 40 Shore A. The material can be handled and formed into different shapes, for example the shape of the nose or face and therefore still has thermoplastic properties. Once cooled it sets into that shape and returns back to its original harness of between 50-60 Shore D.

This property is achieved by applying the desired amount of radiation to achieve some level of crosslinking but not too much. Too much radiation would result in too many crosslinked bonds forming that may make the material too hard and not easily mouldable to the face when heated above its melt temperature or may degrade the material or other mask components. Not enough radiation would result in not enough crosslinked bonds forming and the material would not have sufficient three-dimensional shape memory and would be difficult to handle above its melt temperature, it would lose it shape and be sticky or tacky.

The amount of radiation applied should result in an absorbed does of between 4-40 kGy, or between 6-24 kGy. The Gray (symbol: Gy) is a derived unit of ionizing radiation dose in the International System of Units (SI). It is defined as the absorption of one joule of radiation energy per kilogram of matter

Product dose absorption is dependent on the characteristics of both the electron beam and the product itself. As the electron beam enters the product being irradiated, dose is absorbed in the product. The absorbed dose will vary as a result of the uniformity of the product. Generally, the orientation of products for electron beam processing is chosen so that the thickness through which the electron beam passes is less than the depth where the exit absorbed dose is equal to the entrance dose. For 12 MeV electrons this corresponds to approximately 4 g/cm². The total dose may be applied as a single sided dose or as a double-sided dose.

Electron-beam processing also has the ability to break the chains of DNA in living organisms, such as bacteria, resulting in microbial death and rendering the space they inhabit sterile. E-beam processing has been used for the sterilization of medical products. While sterilization of these products may not be required, and is not the primary purpose for the E-Beam processing, it could be an added benefit as the products can be E-Beam processed in their final sealed packaging.

### Seal Thermoforming and Force Distribution on the Face

When heated to above its melt temperature of 58-60° C., the face shield, or CPAP mask seal, has a hardness of less than 10 Shore A or as low as 20 Shore 00. This is less than a CPAP mask silicone seal that are typically 40 Shore A. Human skin has a typical hardness of 20-30 Shore 00.

This lower hardness, in the mouldable state, causes less compression of the skin on the face when the seal is placed on the face, for fitting and thermoforming, relative to a traditional CPAP mask. The mouldable seal sets once it cools to below its melt temperature of 60° C., into a shape that matches the contours of the users face while causing less deformation or compression of the skin relative to that caused by a traditional CPAP mask silicone seal. Once set the mouldable seal has a hardness of more than 15 Shore D or between 50-60 Shore D and as it is not subject to as much deformation as a silicone seal, therefore it more evenly distributes the forces over a larger area of the face resulting in lower pressure being applied to areas of the face and hence leads to less pressure sores forming on the users face.

As can be seem in FIG. 2, a traditional silicone seal 5 can cause localised point loading on the face, indicated by arrows 24. This point loading can occur in regions of the seal that are thicker, where the facial profile differs greatly from the moulded form of the seal and/or under the side wall of the seal 23. These factors result in the forces being applied to the face, from the silicone seal over significantly less area than the area over which the seal contacts the face.

For example, the approximate significant force contact area of a traditional full-face mask silicone seal is approximately 25 cm². This significant force area, where at least 80% of the force is applied, is less than the total seal contact area 40, as discussed above, and is an estimate of the area where most of the force is applied, for example underneath or close to the vertical walls of the seal or thicker areas of the seal. The approximate contact area of the medium full-face mask face shield is 100 cm², which is 4 times greater than the traditional full-face silicone seal significant force area. Therefore, for the same applied force the pressure applied to the face will be on average 4, or between 3-5, times less while using the face shield or mask of the present invention.

In summary, the significant force area of full-face seal contact is a measure of the higher or peak pressure areas, and the total seal contact area 40 is a measure of the overall or average pressure applied by the mask. This provides two means of assessing pressure area, one being higher pressure areas and the other average pressure areas. The average is more straight forward to calculate as it is a simple measure of the total seal contact area.

### Mask Assembly

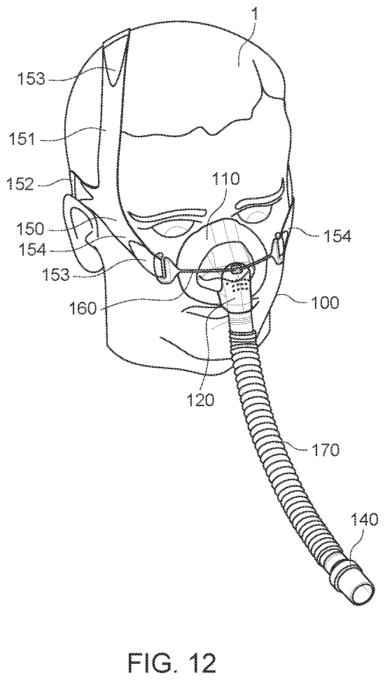

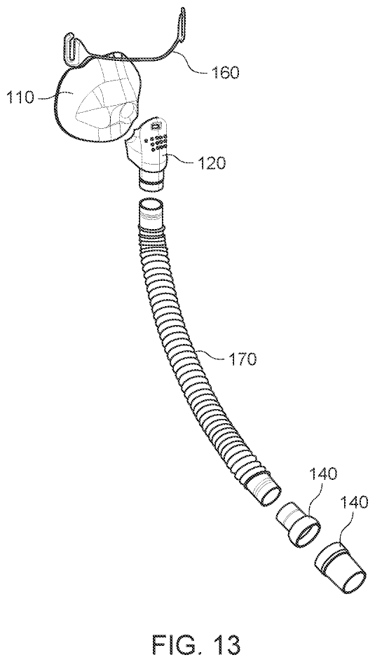

Mask assembly embodiments of this invention, shown in FIGS. 12-21 are directed towards a mask provided with seal constructed from a low melt temperature, hard, thermoformable material, where the seal is connected to a frame. The frame is made from a material that has a higher melting temperature than the seal material. As described in greater detail below, the frame has features such as a headgear connection, vents for gas washout and a connection to a breathing circuit.

As the hard thermoformable seal provides a mask assembly that is inherently stable, it may not need to have the peripheral stabilising features, such as three or four headgear connections located around the periphery of the mask or a forehead support that are typically required to provide masks with stability. As the mask has a hard, rigid seal it cannot easily be pulled sideways as the seal will not collapse or flex like a standard silicone cushion seal.

The frame supports the form of the cushion during the heating and moulding to the face. It also forms a small internal breathing chamber below the nose. The breathing tube connection to the mask frame can be located below the nose, in a downward direction, and does not extend out significantly beyond the tip of the nose, resulting in a very low-profile mask.

Such an arrangement provides a mask that is less obtrusive as it does not have the peripheral stabilizing features, a large breathing chamber that protrudes from the face and a bulky flexible seal. It also has improved air sealing properties and creates less pressure points on the face. This allows users to more freely sleep on their side or partially face down into the pillow without the mask being dislodged by a traditional bulky elbow connection.

Mask assembly 100 is placed on a user's 1 face to create an air seal and is connected via a breathing circuit to a CPAP device in the same general manner as mask 2 of FIG. 1 is depicted. It will be appreciated that the mask assembly as described in the mask assembly embodiments of the present invention can be used in respiratory care generally or with a ventilator, but will now be described below with reference to use in a Continuous Positive Airway Pressure (CPAP) system. It will also be appreciated that the present invention can be applied to various forms of mask assembly including, but not limited to, nasal mask, full face masks and nasal pillow masks that cover the user's nose and/or mouth. The mask may cover the nose and/or the mouth and be available in a range of sizes, such as small, medium and large.

### Nasal Mask Assembly

FIGS. 12-20 show a sixth embodiment, a nasal mask assembly 100 that is provided with a seal 110 constructed from a low melt temperature, hard, thermoformable material. FIG. 12 shows the mask assembly 100 on a user's 1 head covering the user's nose. Mask assembly has a frame 120, a seal 110, headgear 150, a sliding connector 160 or headgear connection mechanism, a short breathing tube 170, and swivel connection joint 140 that connects the mask to the main breathing circuit that is connected to a CPAP device.

### Thermoformable Seal

The seal 110 is formed from a low melt temperature, hard, thermoformable plastic material. The seal material may have a melting temperature between 50-70° C. The thermoformable material is also relatively hard (and hence relatively rigid) when in a set or cooled state, for example it should have a harness of more than 15 Shore D, or 50-60 Shore D. Most CPAP masks use a silicone with an approximate hardness of Shore A 40, that is a significantly softer scale than the minimum value of the Shore D scale. It should also be noted that silicone in not a thermoplastic, it is a thermoset material and cannot be heated and thermoformed from its original shape.

As the seal is not required to curve in on itself, where it contacts the face, to create a seal using air pressure as common silicone seals do, as shown in FIG. 2, it can contour the nose more closely, reducing the size of the breathing chamber and the size of the mask. Conventional mask seals also have vertical walls of approximately 20-40 mm, as measured in the sagittal plane, to separate the rigid frame from the face and to allow for seal conformity to different user's facial contours. As seal 110 is hard it does not need to be supported by a large frame over most of its sealing area, reducing the size of the frame and breathing chamber.

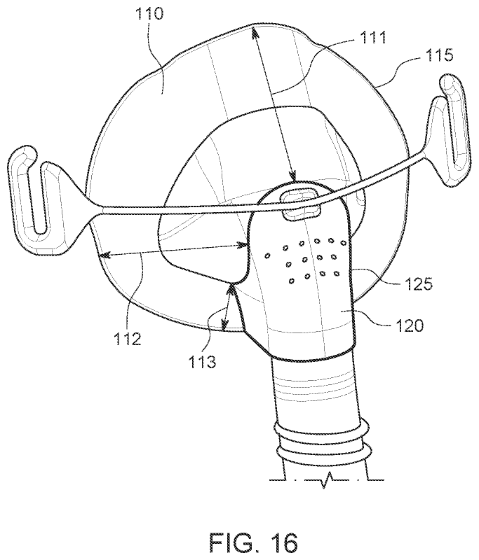

The thermoformable seal 110 extends out, in a radial direction, measured in the coronal plane, from the frame in at least one of the nasal bridge or cheek regions, contouring to these regions forming a low-profile seal formed to match the general contours of the face but is not customised to a particular users face. FIG. 16 shows seal 110 extends out from the frame 120 by 20-40 mm in a superior direction 111 to a seal outer perimeter 115 beyond the frame outer perimeter 125 along the user's nasal bridge region and extends from the frame by 20-40 mm in a lateral direction 112 over the user's cheeks creating a substantial air seal, or air connection, against the face. In the upper lip region, the seal may be substantially covered by the frame, or extend 10-20 mm from the frame in an inferior direction 113. The outer edge of the seal 110 may be generally curved or radiused to improve the comfort on the users face.

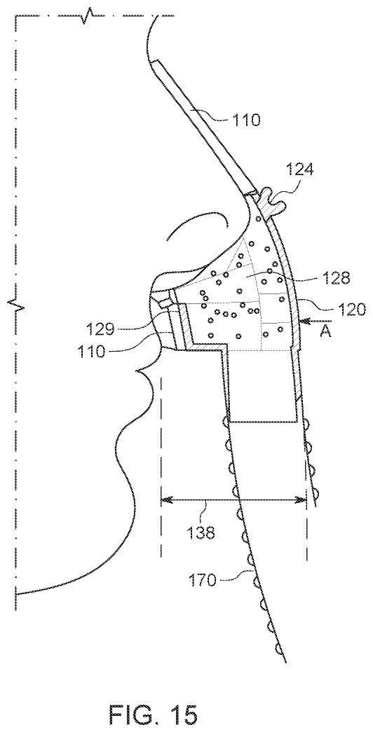

The thermoformable seal 110 is 1-4 mm thick, or between 2-3 mm thick, in the direction that is perpendicular to the face contacting surface of seal 110. FIG. 15 shows the thermoformable seal 110 extending out from the frame in a generally planar manner (as shown in cross section) creating a seal with an aspect ratio of between 5-40, or between 6.67-20 (seal extension from frame/seal thickness). This high aspect ratio creates a thermoformable hard seal that has increased contact area to reduce pressure while being thin in the other direction (2-3 mm) make it practical to heat and thermoform. If the seal was significantly thicker it would be impractical to heat and thermoform to the users face as it would take a long time to heat and cool. Increased cooling time can lead to the user inadvertently moving the seal during the cooling phase leading to unwanted seal deformation and a poor fit to the face.

The outer perimeter of the seal 110, where it contacts the face, projected onto the coronal plane forms a projected seal area. The outer perimeter of the frame 120, where it forms the breathing chamber, excluding any tube connection 127 feature, projected onto the coronal plane forms a projected frame area. The seal projected area of the sixth embodiment is approximately 20 cm² and the projected area of the frame is approximately 10 cm². The ratio of nasal mask seal area to frame area is approximately 2 or in the range of 1.5-2.5. A conventional CPAP nasal mask, for example the HC407 has a projected seal area where it contacts the face of approximately 15 cm² and a frame breathing chamber projected area, e.g., excluding the forehead support, of approximately 20 cm². The ratio of seal to frame area is approximately 0.75. The seal/frame larger ratio of the present invention is possible as the seal is hard and does not need to be supported over a large area by a frame, leading to a more compact breathing chamber and mask.

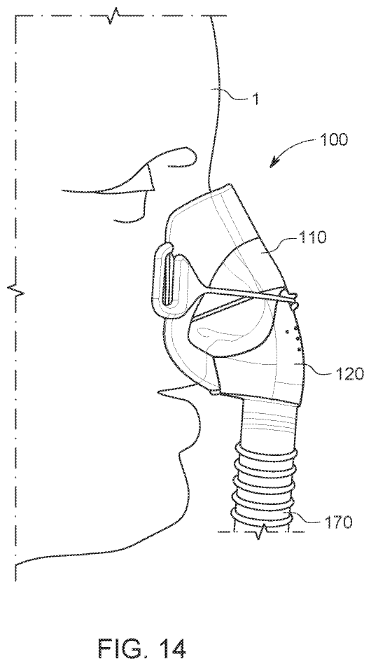

The thermoformable hard seal allows the mask frame 120 to be significantly smaller and may be located substantially below the nose (in an inferior direction relative to the tip of the nose). The above features combine to make the mask very low profile on the face, as can be seen comparing the side view of the conventional mask shown in FIG. 2 to the side view of the present invention in FIGS. 14-15. The less the mask protrudes from the face the more stable it is and the less it obscures the users vision leading to less claustrophobia.

The thermoformable seal 110 may be injection moulded from polycaprolactone as detailed herein.

The Seal 110 is crosslinked after injection moulding, before being customised to a specific users face, to impart shape memory into the seal, allowing the seal to hold its moulded shape, without significant unwanted deformation, thus improving the handling and fitting of the mask, allowing untrained users to fit their own mask. These details and benefits are described above.

The mask can be heated to above 60° C. for example by placing it in hot water, to soften the seal, but not softening the mask frame, in order to place the seal on the users face to form it to the contours of the particular users face. During this process the frame does not substantially soften and acts to provide support to the seal and act as a handle for the heating and fitting process. The seal forms a substantially air tight connection to the users face after being moulded to the shape of the user's face and cooling below its melt temperature or below 50-70° C. Further benefits of mask and fitting process are described in the section.

Once set the mask seal matches the contours of the users face, and applies the mask retention force over a large area of the face, improving the sealing performance and reducing the peak pressure applied to the face by a factor of 3-4 relative to conventional silicone seal masks or to reduce the average pressure applied to the face by a factor of 1.4-2. The basis of this calculation is the same as described in the face shield section.

As the seal is hard and rigid the mask is inherently stable and does not need additional features such as a forehead support or rigid side arms to provide stability.

In a variation of the sixth embodiment, the nasal mask seal 110 may additional extend down from the upper lip to cover the mouth, preventing air for leaking out of the mouth. The seal could extend between 30-90 mm in an inferior direction, over the mouth and/or under the chin to both cover the mouth and provide a chin support, to prevent the mandible from lowering and the mouth from opening. This seal could also extend under the chin while having and opening for the mouth, enabling the user to breathe through their mouth while providing chin support. Additional headgear connections to the lower region of the mask may be added to support the lower region of the mask or the connection point for the headgear may be lowered. This mouth covering variation may also include a non-rebreathing valve for safety as the mouth is covered.

### Thermoformable Seal to Frame Connection

The frame may be injection moulded and then the seal is injection moulded over the frame, this process is called over moulding or insert moulding and can result in a bond between the seal and frame that is chemical, mechanical or a combination of the two. The seal could also be formed separately and then bonded or glued to the frame. The combined frame and seal form a unit that improves seal handling during the heating the thermoforming fitting process. It is not practical to have a separate seal 110 without a bonded higher temperature frame or clip as the seal 110 region that connects to the frame would distort during the user thermoforming process and would not be able to connect to the frame or clip after thermoforming.

The seal may additionally be over moulded with a layer of silicone or EVA, substantially incapsulating the seal, in order to give the mask seal different texture or feel on the face. This version would only be suitable for users that do not have silicone allergies. The layer of silicone or EVA would be relatively thin, in the range of 0.5-2.0 mm and would not be allowed to flex independently of the thermoformable seal 110 material, so seal 110 would still retain it generally rigidity to support itself without the need of an extensive frame to support it over most of its area.

Alternatively, the seal may be over moulded, or bonded, to a connection mechanism, such as a seal clip or cushion clip. The clip can be formed from the same type of high temperature, rigid materials that can be used to form the frame. The combined seal and clip form one unit that can then be connected to the frame. This connection can be permanent, for example it can be glued, welded or permanent clipped together or it can have a releasable snap fit connection, enabling users to easily remove the seal from the rest of the mask assembly for cleaning or changing to a different size of seal or even a different type of mask, for example changing from a nasal mask to a nasal pillow or full face or any combination of these. U.S. Pat. No. 10,272,218, incorporated entirely by reference here, describes a suitable cushion clip, for example bridging portion 50 or clip 442, for connecting the cushion to the frame or mask body.

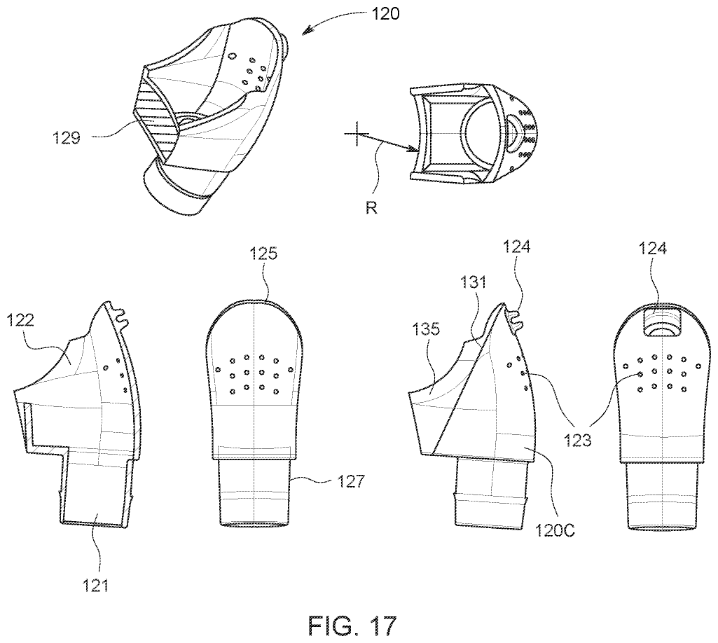

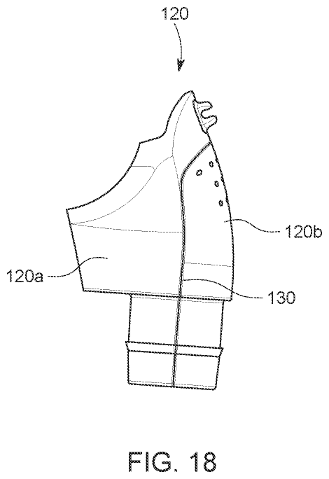

For example, FIG. 17 shows frame 120c and clip 135 that are connected along joint 131. This will also enable easier over moulding of the frame with the seal. FIG. 18 shows frame 120 formed from two components, lower frame section 120a and upper frame section 120b. This design allows for easy over moulding of lower frame section 120a with seal 110. After over moulding upper frame section 120b is then connected along joint line 130 using any known permanent or removable connection method. Frame 120c and clip 135 can also be considered to be two components of the frame, as clip 135 provides some of the functions of a frame, such as support to the seal 110 during thermoforming to the user.

### Frame

FIGS. 16-20 show mask assembly 100 has a frame 120 structured to maintain the seal 110 in an operative position with respect to the patient's face in use and while being thermoformed to the users face. The frame 120 is constructed e.g., injection moulded, from a rigid material (e.g., Sabic Lexan HP2 polycarbonate or Eastman Tritan Copolyester) that has a melting temperature that is higher than that of the thermoformable seal material or above 100° C. Polycarbonate and Triton are also suitable as they naturally form a bond, without the use of additional adhesives or treatments, when over moulded with PCL. The frame has a general wall thickness of about 1-2 mm, e.g., 1.5 mm. As shown in FIG. 15, the frame 120 defines a breathing chamber 128 (indicated by the doted region), or cavity, adapted to receive the patient's nose and/or provide air communication to the patient via outlet opening 122. The frame includes a vent arrangement 123 for gas washout.

Using a frame material that has higher melt temperature allows the combined frame and seal unit to be placed in boiling water, or hot water between 60-100° C., without melting the frame, enabling the frame to provide support to the seal and providing a rigid component that acts has a handle for the user in the heating and thermoforming fitting process.

The user may hold frame by the tube connection 127 and/or the lateral side walls of the frame, that form a handling region 132 of the frame the does not need to be entirely submerged in hot water while allowing seal 110 to be fully submerged in order to raise its temperature to or above 60° C., in order to bring the seal to a thermoformable state.

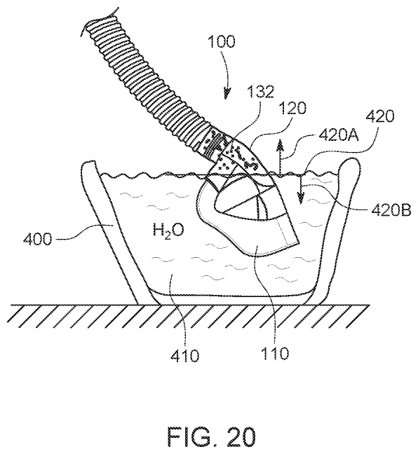

FIG. 20 shows the combined frame and seal unit being placed in a bowl 400 of hot water 410. Handling region 132 extends out of the water and is located on the upper side 420A of plane 420 that is defined generally by the surface of the water. The entire region of seal 110 that is required to be softened for thermoforming to the face, is located on the lower side 420B of the plane 420, in the water. Alternatively, other structures may connect to the frame and/or seal clip that are made from high temperature materials, to assist the user heating the seal, that can be removed before use. The flexible region of tube 170 is not considered to be part of the handling region, as it cannot withstand temperatures of 100° C. without being damaged.

### Breathing Circuit or Tube Connection to Frame

The frame includes an inlet opening 121 and tube connection 127 adapted to receive a short flexible breathing tube 170 or the main breathing tube 4 directly. Alternatively opening 121 and/or tube connection 127 may connect to an elbow and/or a swivel joint. FIG. 15 shows opening 121 is located below the nose and tube connection 127 may protrude down from the mask assembly in an inferior direction. Alternatively, there may be one or two tube connections 121 that may protrude generally in one or two lateral directions, and may include one or two breathing tubes being connected. Furthermore, the tube connection may protrude up in a superior direction and pass over the forehead, or an elbow may connect to the frame, allowing the tube to be directed in a number of directions.

Opening 121 is located at least partially, or substantially, in a posterior direction relative to the tip of the nose. This reduces the amount the mask assembly protrudes out from the face, in an anterior direction. Many CPAP masks have elbows that protrude in an anterior direction from the frame, as can be seem in FIG. 2 that shows protrusion 25. For a conventional nasal mask such as the Fisher & Paykel Healthcare HC407 or Zest nasal masks, generally depicted in FIG. 2 and in U.S. Pat. No. 10,272,218, protrusion 25 can be about 70-80 mm from the central upper lip contacting region of the seal to the outer most part of the elbow or mask, in the anterior direction. This protrusion can cause the mask to contact bedding when the patient sleep on their side or face down, and this can cause the mask to dislodge and leak. As shown in FIG. 15 the equivalent protrusion 138 on the nasal mask described herein may be 25-40 mm or less than 40 mm. The large distance 25 for the convention mask, shown in FIG. 2, also allows the breathing tube forces to create increased torque on the mask, leading to movement and leaks or the headgear being tightened to counter this leading to increased pressure on the face and pressure sores. As the mask described herein decreases this distance 138 to a value to around half that of standard masks this torque is also halved leading to less mask movement, leaks and/or pressure applied to the face.

### Headgear and Headgear Connection to Frame

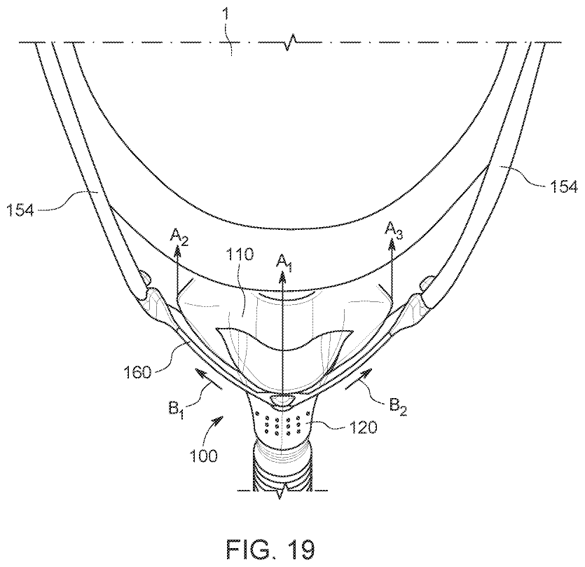

FIGS. 12 and 19 show mask 100 includes headgear 150 for securing the mask to the user's head. The headgear may include lateral straps 154 that are connected to the frame directly or via other mechanisms described below. Lateral straps pass over the cheek region and connect to an upper rear strap 151, and a lower rear strap 152. Lower rear strap 152 may pass above or below the ears. The headgear is made from breathable fabric and/or foam laminate such as Breath-o-Preen or similar materials know in the art. While one side of the headgear is shown in FIG. 12, the headgear is located on both sides of the head and the side not shown is a mirror image of the side that is shown. The headgear straps may include Velcro™ brand or style hook and loop connectors 153 for adjusting the length of each strap to suit the user. Left and right lower rear straps 152 and upper rear straps 151 may connect to each other at the back of the head/neck and at the top of the head respectively via eyelets or other known means. FIG. 12 shows headgear with one lateral strap on each side of the face as this enables the user to easily adjust the tension on each side and once, with one hand on each side. However, the nasal mask and in particular a full-face mask, may have two pairs of lateral straps. In this case, the lower of the two lateral straps would pass lower on the face and below the ear. The headgear straps should connect together to form one headgear unit for ease of adjustment, handling and washing.

FIGS. 12, 17 and 20 show frame 120 includes one headgear connector 124 that removably connects headgear 150 to the frame either directly, for example via open or closed eyelets in the frame, for example, as see in the forehead support of the FPH Zest nasal mask. Alternatively, the headgear 150 may connect to the frame via a sliding connector 160 or other headgear connectors such a clips or snap fit connections known in the art. The use of sliding connector 160 provides a low-profile connection mechanism when compared to eyelets or snap fit connectors, this reduces contact with the patients bedding or forces generated form such contact that can lead to mask movement. U.S. Pat. No. 6,662,803, incorporated here entirely by reference, discloses a suitable sliding strap 200 and engagement clips 202. In addition, a double sliding connection may be used, for example as seen in the FPH HC431 full face mask. One sliding connection can be located above centre of seal contact area, as calculated in the coronal plane, and the other below it to provide additional stability, such as design can be used in a nasal mask or full-face mask embodiment.

Alternatively, connector 160 need not have a sliding connection, but may have the same general low-profile connection and form.

Headgear connector(s) 124 are located near the centre of area of the seal, as calculated in the coronal plane, which is generally over the tip of the nose for a nasal mask, as shown in FIGS. 14-15. The nasal mask frame may have only one or two headgear connectors. Where conventional nasal masks may have three or four headgear connection points located around the periphery of the frame, as is required to stabilise nasal masks with flexible silicone seals. Headgear connector 124 may be located on the midline of the mask frame rather than being a pair of connectors located laterally, as the mask is inherently stable as described below.

As shown in FIG. 19, headgear straps 154 create two force vectors B1 and B2, that results in one force vector A1 acting on the frame, in the middle of the mask. Vector A1 is intern transferred from the frame into to two force vectors A2 and A3 acting through the seal 110 onto the face. In use, vector A can rotate to act more to one side of the mask, due to patient movement, causing vectors A2 and A3 to vary from each other in magnitude. In a mask with a conventional silicone seal this would create an unstable mask and lead to leaks when, for example, A3 becomes larger than A2 leading to the side wall of the flexible silicone seal, in contact with the face at A3, to collapse causing leading to further mask movement and a leak in the area of A2, that has reduced in magnitude and reduced sealing force. However, as the mask described herein has a rigid seal, its seal does not collapse when A3 becomes larger than A2, creating a more stable mask with improved sealing performance. This has been simplified for illustration as acting in the transverse plane, however it will be understood that similar vectors to A2 and A3 will also be generated in the sagittal plane.

Connecting the headgear to the mask frame, near the tip of the nose, or on the midline, is advantageous when compared to connecting the headgear to the periphery of the frame or seal, near the surface of the face in the cheek region. This is because force vector A1, acting near the centre of seal area, is more likely to act in a posterior direction within the outer periphery of the seal 110, leading to inherent stability of the mask with a hard thermoformable seal.

A further advantage is created as headgear vectors B1 and B2 act in more of a posterior direction creating a higher magnitude sealing force vector A, when connected near the tip. When connected near the surface of the face, for example, in the cheek region, the headgear straps create vectors that act more in a lateral direction, as they have to curve around the cheeks changing the angle of connection to the mask, resulting in higher headgear tension forces being required to produce force vector A with the same magnitude compared to the mask described herein that connects the headgear distant from the surface of the cheek region. Connecting near the cheek region produces a mask with less stability and poorer sealing performance relative to a central connection described in this embodiment. In addition, including headgear connectors in the seal may lead to poor thermoforming to the face in the region resulting in discomfort and/or leaks between the seal and the face.

Additional suitable headgear and headgear to mask base (frame) connection designs are disclosed in U.S. Pat. No. 9,320,866 ('866), incorporated here entirely by reference, for example headgear 21 that includes curved and elongate member 34 and its connection to the mask base 22, as well as headgear 300 and that of the ninth full face mask embodiment. The mask described herein may not need the stabilising features of '866 however cured and elongate member 34 and its equivalent versions may be useful in changing the headgear vectors, directly them away from the eyes and allowing all rear straps to pass over the top of the ears for ease of placing the mask assembly on and off the face for the nasal and nasal pillow versions.

### Stabilizing Mechanism

FIG. 15 shows the frame 120 includes an upper lip support 129 that is structured to engage the upper lip in a region 57 between the base of the nose and the mouth. The lip support may also be structured to engage the upper lip between the lateral extremities of the left and right alar.

The upper lip support 129 may engage the upper lip directly or may be covered by the thermoformable seal 110 material, as shown in FIG. 15, or another substance, either way the upper lip support of the frame will retain is integrity during the thermoforming process and act directly or indirectly to provide support and a reference point to the face as the relatively thin layer of seal 110 in this region cannot deform significantly as it is covered by the upper lip support 129.

The upper lip support is designed to provide a reference point for the mask assembly during the thermoforming fitting process by transferring forces through the upper tip to the patient's maxilla. This stabilises the mask assembly in the correct posterior-anterior location and reduces rotation in the sagittal and coronal planes. The upper lip support may be contoured to the shape of the upper lip, for example, it may be concave in shape and may include a recess shaped to accommodate the nasal septum, as indicated by radius R in FIG. 17. When thermoforming the mask seal 110 to the patients face the seal is in a softened/rubbery state and may not provide the patient with positive feedback about the correct location of the mask assembly on the face, this could lead to the mask assembly being incorrectly orientated on the face. The lip support over comes this issue by providing a positive reference point for the location of the mask assembly. It is also located close to tube connection 127 that acts as a useful place for the patient to hold during the thermoforming process, allowing the user to push the mask assembly in a posterior direction while the seal cools and set into shape.

Alternatively, the frame 120 may include an alternative to the lip support, such as a cheek support, or nose tip or nose bridge support, that contacts the user in the cheek, nose tip or bridge region, providing the same function as the lip support, that is a reference point for fitting the mask, using the cheek bones instead of the maxilla. Furthermore, temporary structures such as forehead supports or rigid side arms could be used as reference points during fitting and could be removed after the fitting process has been completed. The frame 120 includes one of the following combinations (a) an upper lip support 129 only, (b) an upper lip support 129 and a cheek support, (c) an upper lip support 129 and a nasal bridge support. Support structure should not include all three of these features as due to facial variation amongst users these points will vary resulting in fitting difficulties as the rigid frame will not match these land marks on many users.

### Gas Washout

FIG. 17 shows the frame 120 includes a vent arrangement 123 for gas washout from the breathing chamber 128 or mask. The vent may consist of one or more openings or holes that allow gases to vent out of the mask. These holes may be drilled, laser cut, moulded into the frame, or be an insert that is placed into an opening in the frame. The holes can be 0.5 mm-1.0 mm, or between 0.65 mm-0.85 mm in diameter, or non-round openings with equivalent area, there should be between 25-50 holes (not all holes are shown figures). This large number of small holes reduces the noise levels and draft caused but the gas venting from the mask. The holes should be 1-2 mm deep and pass from the inner surface of the frame to the outer surface of the frame.

The openings or holes may be additionally covered with a filter medium to further diffuse and quieten the gas vent flow and/or to filter water droplets, aerosols, bacteria and viruses from the venting air, reducing contamination of the surrounding environment that may affect others. The filtering of the vented air will benefit both respirator masks applications as well as industrial and personal protection mask applications. U.S. Pat. No. 6,662,803, discloses suitable outlet vent designs including apertures 302, a frame member 306 and filter medium 308.

### Alternative Nasal Mark

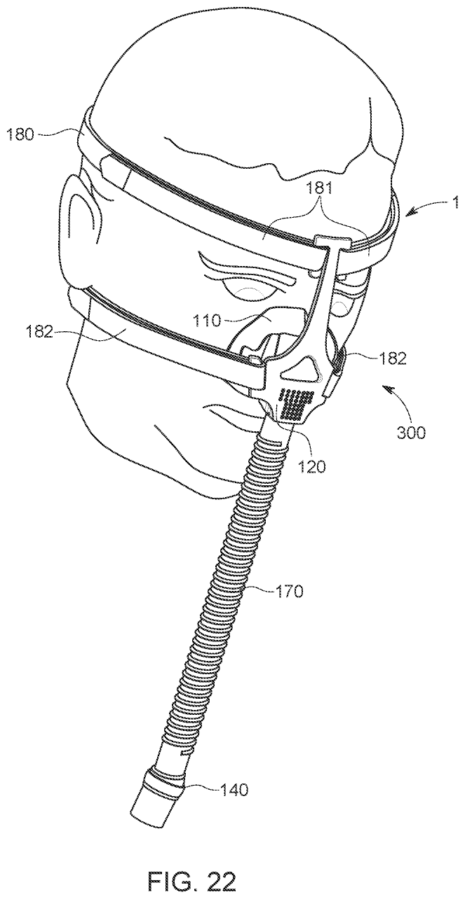

FIGS. 22-26 show a variation of the sixth embodiment, nasal mask 300 with a four-point headgear connection to frame 120. FIG. 22 shows headgear 180 may have a pair of upper straps 181, passing over the user's 1 forehead, and lower straps 182, passing over the user's cheek region, that connect to headgear connectors 124 located on mask frame 120. Alternatively, there may be one or three upper straps 181. Headgear connectors 124 maybe in the form of eyelets, hooks, a glider or other headgear connectors such a clips or snap fit connections known in the art

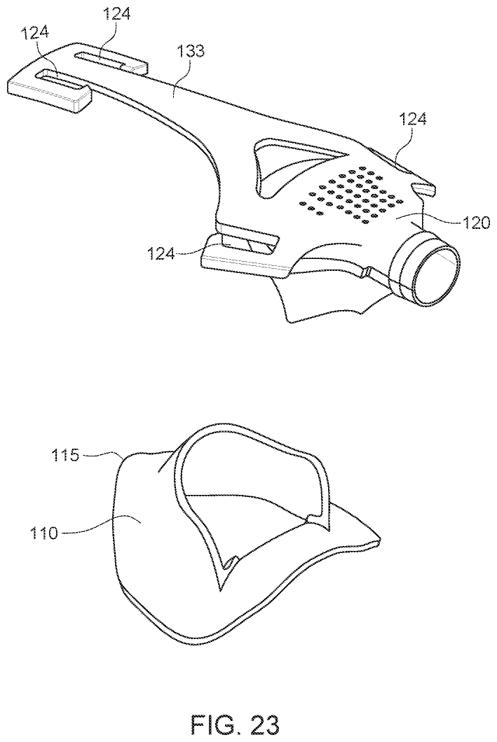

FIG. 23 shows seal 110 comprises the same general design as the sixth embodiment made from the same thermoplastic material that is cross linked after being injection moulded and all have a melting temperature of between 50-70° C., or between 40-80° C., or for example a melt temperature of 60° C. Design aspects of this embodiment also be applied to the other embodiments of this specification. Seal 110 extends out from the frame 120 by 20-40 mm in a superior direction 111 to a seal outer perimeter 115 beyond the breathing chamber outlet perimeter 126 along the user's nasal bridge region.

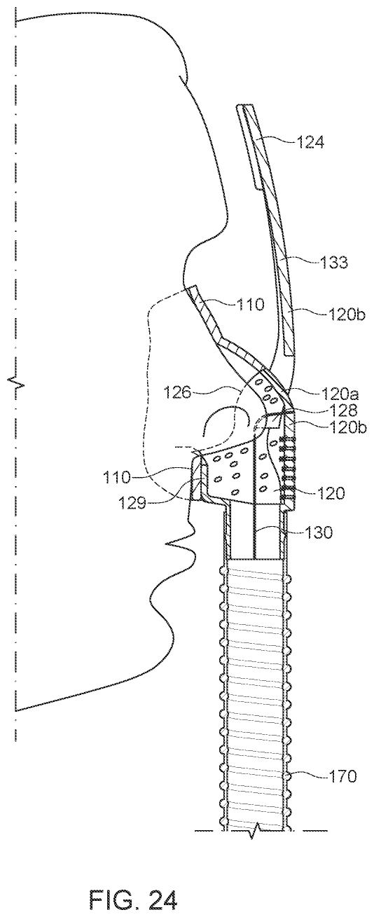

FIG. 24 shows frame 120 extending over the tip of the users nose and near the user's alar in order to support seal 110 and hold seal 110 away from the soft tissue regions of the nose including the alar during the seal thermoforming process where it is customised to a user's face. Supporting seal 110 away from the soft tissue regions can reduce soft tissue movement or compression that may lead to narrowing of the nares that may lead to breathing restrictions. As a frame with alar support may be wider to provide support near the alar, seal 110 may only extend 10-30 mm laterally 112 from the breathing chamber outlet perimeter 126 near the alar region. The design of masks 100, 200 and 300 result in masks where the seal to face contacting region, that is the seal region performing a sealing function in contact with the face, is substantially located outside of the frame perimeter 125 or breathing chamber outlet perimeter 126, as projected onto the coronal plane, creating a low profile mask and reducing the pressure lifting the mask off the face as the net projected area is reduced, reducing headgear strap tension and improving comfort.

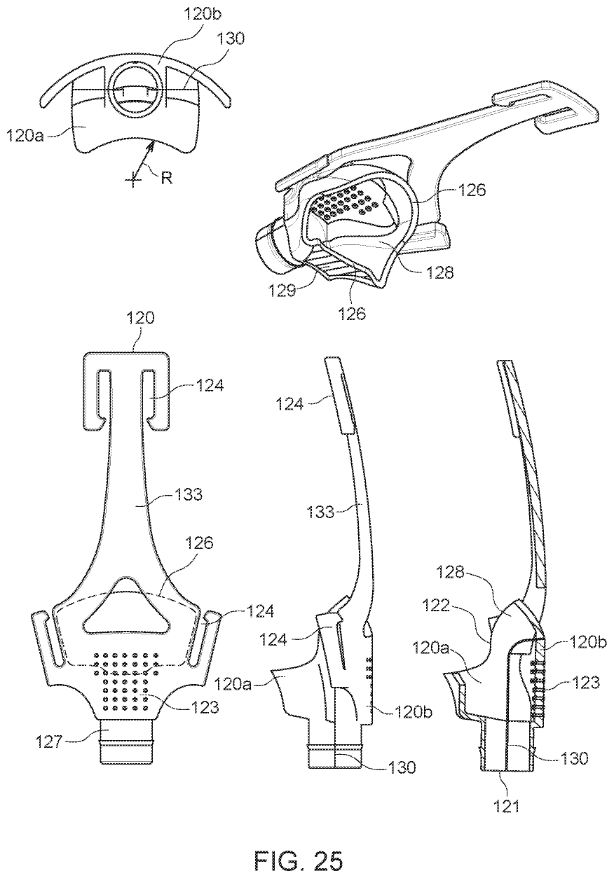

FIG. 25 shows frame 120 has a forehead support 133 extending from the breathing chamber 128 region of the frame up to the user's forehead region for connection to the upper headgear straps 181. Having upper and lower straps allows for independent adjustment of the fit to the face in the upper and lower regions of seal 110. Forehead support 133 extends beyond breathing chamber outer perimeter 126 of the frame breathing chamber 128. Lower headgear connectors 124 may also extend out from outlet perimeter 126 of the frame breathing chamber 128.

It should be noted that calculations relating to the projected area of the frame breathing chamber, or frame outer perimeter, should exclude the forehead support 133, headgear connectors 124 and tube connection 127, lib support 129, or other features where they extend beyond breathing chamber 128 outlet. As frame 120 of mask 100, shown in FIG. 16, does not show any features extending from the breathing chamber outlet, apart from connector 127, and lip support 129, the outer perimeter of the frame minus connector 127 and lip support 129, is the same as the frame breathing chamber outlet in FIG. 16. FIG. 25 shows breathing chamber outlet perimeter 126 of mask 300 is defined by the frame outlet opening 122. Breathing chamber outlet perimeter 126 is also the region of the frame that connects to seal 110, excluding any overlap such as lip support 129 overlap with seal 110.

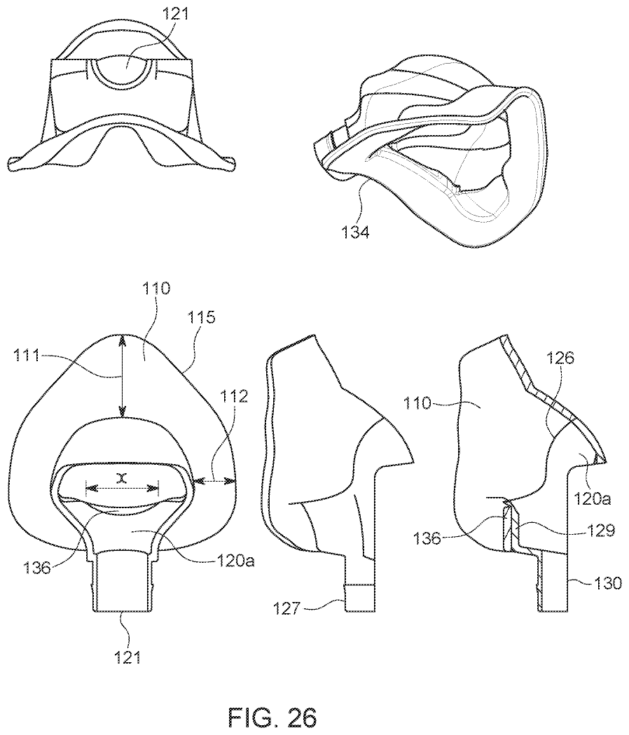

FIG. 26 shows cushion module 134 comprised of seal 110 and lower frame 120a being permanently attached to each other. FIG. 25 shows bias flow holes 123 are shown located in upper frame section 120b. Bias flow holes 123 and/or inlet open 121 could alternatively be located entirely in lower frame section 120a allowing lower frame section 120a and seal 110 to form a cushion module leaving upper frame 120b to removably connect headgear 180 to the cushion module. In a second variation bias flow holes 123 and inlet opening 121 may be formed entirely in upper mask frame 120b, allowing removable connection of the cushion module from the headgear and breathing tube in a similar manner shown in FIG. 17 that shows upper mask frame 120c. In a third variation bias flow holes 123 may be formed in lower frame section 120a and tube connection 121 may be formed in the upper frame 120b. In a fourth variation tube connection 121 may be in the form of an elbow, connected to upper frame 120b, the bias flow holes 123 may be located in the elbow, upper frame or lower frame. Providing and elbow allows for optional positioning of the tube. Cushion module 134 may be provided in a range of sizes or styles to suit different users and may all connect to a common upper frame 120b or shroud. Different upper frames 120b or shrouds could also be provided to one or more cushion modules that provide different features such as different sized upper frames, shrouds or differing numbers and styles of headgear connection, such as one, two three, four or five connection point headgear or frames with or without forehead support or different colours to meet user preferences.

Frame 120a may have a mask logo and/or company brand over moulded onto it during the seal 110 moulding process. This would form a logo or brand in the same material and hence colour as the seal. This logo could be moulded into a recess, or embossed region of frame 120a. PCL is often white or coloured and this will stand out against a clear frame. During the heating process the logo or brand will go above the melting point of the polycaprolactone and become transparent or semi-transparent providing further visual indication that the seal is ready to fit to the user's face. Other symbols or messages such as ‘ready’ could be conveyed to the user in the same manner as the logo or brand.

FIG. 26 shows seal lip extension 136 is a region of seal 110 that extends beyond the rigid frame lip support 129, in a superior direction, towards the user's nose. Seal lip extension 136 is located on the midline and have a minimum width (x) of 8 mm, as measured in the lateral direction, or can be a greater width extending out to the side walls of the frame 120. For example, seal lip extension could be in the range of 8 mm-48 mm wide. Seal lip extension 136 allows for the seal to contour, during the thermoforming process, to the user's lip where it transitions to the nasal septum, reducing contact between this region and the rigid frame lip support 129 that does not thermoform to the contours of the users face, improving comfort should the mask be pulled in a superior direction towards the nose. Seal lip extension 136 extends from frame lip support 129 a minimum of 2 mm or preferably at least 5 mm in a superior direction. Alternatively frame lip support 129 may not be located on the midline at all, it may be two regions located either side of the midline allowing seal lip extension 136 to be located between the two frame lip supports 129, improving contouring of seal 110 along the midline of the upper lip.

The thickness of seal 110 may vary, for example the seal region that is located near the users alar or other soft tissue regions around the tip of the nose, may be thinner, for example in the range of 0.3 mm-1.2 mm. This allows the seal to fit to this region while reducing pressure applied to the alar reducing narrowing of the nares during fitting that could lead to breathing restriction.

The seal outer perimeter 115 could also have a reduced thickness in order to allow the perimeter to flex slightly reducing pressure on the user's face at the seal perimeter caused by mask movement. The average thickness near seal outer perimeter could be reduced to between 0.3-1.2 mm over the region 2-5 mm from the perimeter of the seal. The region 0-2 mm from the perimeter may also be reduced or may be thicker than 1.2 mm to avoid a sharp edge forming at the seal perimeter. The thinner region of between 0.3-1.2 mm, located 2-5 mm form the perimeter, will allow the perimeter to be thicker while still providing flex for comfort.

Seal 110 or frame 120 may have curved and elongate members, formed from the thermoformable seal 110 material, such as polycaprolactone, extending from the seal or frame in order to connect to headgear. Examples of suitable curved elongate members 34 are disclosed in U.S. Pat. No. 9,320,866 ('866). Curved and elongate members may be formed integrally with the seal or frame over moulding process or may be mechanically or chemically connected. Curved and elongate members can have a region that has a thickness of less than 2.0 mm near its connection to the seal or frame to allow the member to flex to the facial profile of individual users, for example over the cheek or forehead region. Alternatively, curved elongate members may not have a thin section for flexing as they can be thermoformed to each individual user. Such members will also be cross linked for shape memory providing the same advantages as disclosed for seal 110. The use of curved elongate members for connection to headgear can provide additional stability to the mask.

Headgear connectors 124 may be located in seal 110 or in curved elongate members and may be formed from a material that has a melt temperature above 100° C., such as the materials used to form rigid frame 120. This enables headgear connectors 124 to maintain their form, for connection to headgear or headgear clips etc., during heating and thermoforming to the users face. Headgear connectors could be injection moulded from rigid materials and then over moulded by the low temperature thermoplastic material that forms seal 110 during the seal 110 or elongate member injection moulding process. Alternatively, they could be mechanically or chemically connected. Having headgear connectors on seal 110 or elongate members provides a lower profile connection between headgear 180 and seal 110 or frame 120. Lower headgear connectors 124 may be located in seal 110 or elongate members while upper headgear connectors 124 may be located in frame 120 as they can be connected to the forehead support 133 in a low-profile manner. Alternatively, mask 300 may also only have a pair of lateral headgear straps 154 as shown in mask 100 in which case headgear 150 may be used on mask 300 and forehead support 133 may not be present.

### Full Face Mask

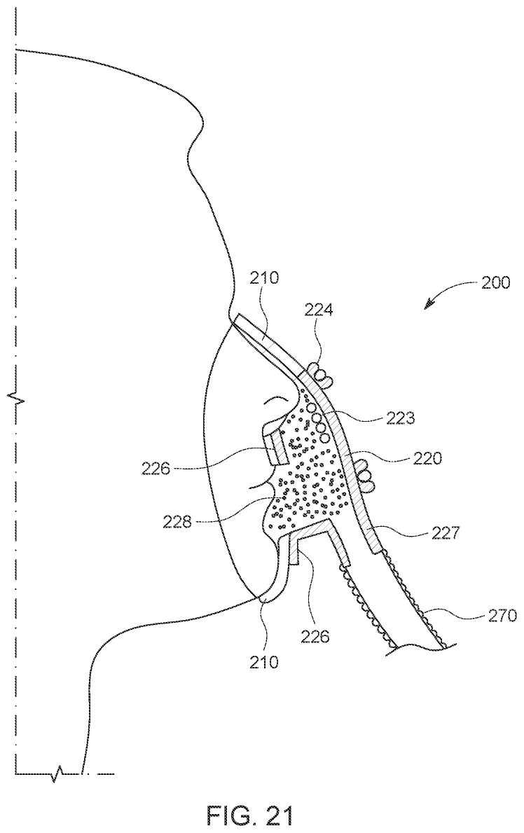

In a seventh embodiment, there is provided a full face mask 200 as shown in FIG. 21 comprising a full face mask 200 that has the same general features, materials and benefits as described in the nasal mask embodiment, such as a low melt temperature, hard, thermoformable seal 210, a frame 220 constructed from a material with a higher melt temperature than the seal material, with changes necessary to create a breathing chamber that additional communicates with the users mouth, vent arrangement 223 and headgear (not shown). It will be appreciated, by those skilled in the art, that the general description of the nasal mask embodiment design, function and benefits also apply to the full-face mask embodiment.

Thermoformable hard seal 210 will extend out from the frame in the same manner in the nasal bridge and cheek regions and additional it will extend 20-40 mm from the frame in a lateral direction either side of the mouth and 20-50 mm in an inferior direction over the user's chin. In the chin region it may go under the chin to act as a chin support. The general contact area of the face may be similar to that of the full-face mask 30 face shield shown in FIGS. 4-7, the second embodiment described, or may have a smaller outer perimeter, similar to outer perimeter of full-face seal contact 38, as it does not need to be sized larger that a full-face mask seal.

Frame 220 extends in an inferior direction relative to frame 120, of the nasal mask, to create a breathing chamber 228 that communicates with the users nose and mouth. Frame 220 also extends in a lateral direction to substantially cover the user's mouth, for example frame 220 should be between 40-80 mm wide in the region covering the user's mouth.

The outer perimeter of the seal 210, where it contacts the face, projected onto the coronal plane forms a full-face seal projected area. The outer perimeter of the frame 220, that defines the breathing chamber, excluding any tube connection 227 feature, projected onto the coronal plane forms a projected full-face frame area. The seal projected area of this full-face embodiment is approximately 50 cm² and the projected area of the frame is approximately 20 cm². The ratio of full-face seal area to frame area is approximately 2.5 or in the range of 2.0-3.0. A conventional CPAP full facemask has a projected seal area where it contacts the face of approximately 50 cm² and a frame projected area, excluding the forehead support, of approximately 50 cm². The ratio of seal to frame area is approximately 1.0. The seal/frame larger ratio may be possible as the seal is hard and does not need to be supported over a large area by a frame, leading to a more compact breathing chamber and mask.

The frame 220 may include an upper lip support 226 to act as a reference for fitting, alternatively it may include a cheek support or chin support region during the user thermoforming process.

The frame 220 may be formed in one or two components, as detailed in the nasal mask embodiment.

The full-face mask 200 may also have a non-rebreathing valve (NRV) to vent to atmosphere in the event of a pressure supply disruption.

The full-face mask frame 220 can also have upper and lower headgear connection points 224 to accommodate pairs of upper and lower lateral headgear straps. These may be located on the midline of the frame above and below the centre of seal area in the coronal plane. The headgear may have one or two pairs of lateral side straps. Given the mask is inherently stable it is possible to provide a full-face mask with only one pair of lateral side straps, connected to frame 220 near the centre of area of the full-face seal, that would be more minimal for the users.

The full-face mask 200 may be very low profile relative to conventional silicone full face mask and have the same stability and general benefits described in the nasal mask embodiment. In another variation, the mask may take the form of a total face mask, such as the FitLife Total

Face Mask from Philips. In this case the frame would be formed in a clear material to cover the user's mouth, nose and eyes. The thermoformable seal would extend from the frame to the face and seal on the user's forehead, side of the face and/or cheek and the chin. There may be an additional seal, passing over the user's nasal bridge and cheek region, to separate the nose and/or mouth breathing chamber from the chamber over the user's eyes. This will reduce fogging or condensation forming on the frame of mask in a region that may obscure the user's vision.

### Nasal Pillow Mask

In an eighth embodiment, there is provided a nasal pillow mask configured to seal substantially under the nose, around the nares. It will be appreciated, by those skilled in the art, that the general description of the nasal mask embodiment design, function and benefits also apply to the nasal pillow mask embodiment.

An example of such as mask is he FPH Opus 360 nasal pillow mask details in '866. In this eighth embodiment, the silicone nasal pillows of '866 are replaced with a thermoformable hard material, as detailed in the nasal mask embodiment described herein. In this embodiment seal 110 would contact and form a seal around the user's nares. The thermoformable nasal pillow seal may from one seal around both nares, for example like the FPH Evora nasal mask or it may seal around and or in each nares like the Opus 360 nasal pillow mask. This embodiment may have a frame lip support to provide reference when thermoforming as described in the nasal mask embodiment. The seal of this embodiment will not extend over the user's nasal bridge region but it may extend over cheek region to stabilise the mask. The headgear designs of the nasal pillow mask embodiments of '866 may also be applied to the nasal pillow mask described herein, such as the headgear and curved elongate member, as the small nasal pillow mask may benefit from additional stability provided by these features.

It will result in a very low profile and stable mask that as the associated benefit described in the nasal mask embodiment, such as improving sealing performance, reducing pressure sores and preventing silicone allergies.

Other aspects of the nasal mask embodiment can be applied to this embodiment, such as the materials, crosslinking, tube connection, gas washout, headgear, sizing options and benefits etc.

### Personal Protection Equipment Mask

In a ninth embodiment, there is provided a personal protection mask used in industrial and healthcare worker application applications, such as 3M Half Facepiece Respirator 7000 series. Personal protection masks are also used by the general public or healthcare workers to filter out city air pollution or air borne viruses in public and healthcare settings.

These masks may, or may not, be connected to a flow device but rely on the user to drive flow in an out the mask as they breath. They may have one, two or more openings in the form of frame inlet opening 121. For example, one may allow for air to enter the breathing chamber and another frame opening to allow exhaled air to exit the breathing chamber to the surrounding atmosphere, acting as an outlet opening. The frame inlet opening 121 may have a one-way valve to only allow air to pass into the breathing chamber and the outlet opening may have a one-way valve to only allow air to pass out of the breathing chamber. The inlet and outlet openings may also be connected to filters to clean the incoming and/or out-going air. The filters can be removed for replacement, cleaning or selecting different types of filters for specific applications, such as filtering bacterial, viruses, organic compounds, city air pollution or other industrial contaminants. The filtering of the outlet opening for aerosols, water droplets, bacterial and virus can protect others that may be nearby from bacterial and/or viral infections that the mask wearer may have, such as COVID-19.

They typically have a rigid frame, headgear and a rubber or silicone seal that engages the face. It will be appreciated by those skilled in the art that these flexible seals can be replaced with the hard thermoformable seal described herein and other aspects described may also apply such as the frame and headgear.

In addition, the mask may have a clear visor that extends up from the frame in a superior direction in order to cover the user's eyes to prevent the eyes being a pathway for infection. The visor may be part of the frame, that is formed as one unit during injection moulding, or it may be connected permanently or removably to the frame or other mask components, so the user can optionally use the visor when required.

In another variation, the visor may come into contact with the users face, around the eyes to seal the eyes from the environment. The face contacting region of the visor may additional have a foam seal or a hard thermoformable seal, as described in this specification. In another variation, the mask may take the form of a total face mask, such as the FitLife Total Face Mask from Philips. In this case the frame would be formed in a clear material to cover the user's mouth, nose and eyes. The thermoformable seal would extend from the frame to the face and seal on the user's forehead, side of the face and chin. There may be an additional seal, passing over the user's nasal bridge and cheek region, to separate the nose and/or mouth breathing chamber from the chamber over the user's eyes. This will reduce fogging or condensation forming on the frame of mask in a region that may obscure the user's vision.

The same benefits also apply, for example a more compact lower profile mask with improved seal and comfort for the user. This will result in a mass manufactured, affordable, customisable personal protection mask.

### Advantages

Selected advantages of the face shield, patient interface and methods and uses thereof may include:
Provision of a fully customised seal;
The versatility to provide a face shield both as an OEM part or after market;
Minimising the need for firm or tight strapping since the seal between the patient interface and face is superior;
Minimising or preventing leakage from the patient interface particularly when the patient moves;
Minimising or preventing pressures sores since the pressure on the patient's face is even and highly customised to the patient and there are no localised pressure points;
Dead space in a patient interface frame may be minimised hence reducing frame bulk;
The patient interface is generally more stable than art solutions;
The face shield is not manufactured from silicon and provides a barrier to any silicon parts that may be present hence avoids silicon allergy issues.

The embodiments described above may also be said broadly to consist in the parts, elements and features referred to or indicated in the specification of the application, individually or collectively, and any or all combinations of any two or more said parts, elements or features. Further, where specific integers are mentioned herein which have known equivalents in the art to which the embodiments relate, such known equivalents are deemed to be incorporated herein as if individually set forth.

Aspects of the face shield, patient interface and methods and uses thereof have been described by way of example only and it should be appreciated that modifications and additions may be made thereto without departing from the scope of the claims herein.

What is claimed is:

1. A patient interface comprising:
a rigid frame defining a breathing chamber configured to receive pressurized respiratory gas; and
a thermoformable seal coupled to the frame and configured to form a sealing interface with a user during use;
wherein the thermoformable seal comprises cross-linked polycaprolactone, the cross-linked polycaprolactone being directly connected to the rigid frame and initially provided in a non-customized shape;
wherein a substantial portion of the facial-contacting portion of the thermoformable seal is positioned beyond the outer perimeter of the portion of the rigid frame forming the breathing chamber, in a cheek region or a nasal bridge region, as measured in the coronal plane;
wherein the thermoformable seal softens when heated to a temperature of about 50-70° C.;
wherein the cross-linked polycaprolactone is configured to retain at least part of the non-customized shape of the thermoformable seal when heated above the softening temperature;
wherein the cross-linked polycaprolactone is configured for direct contact with the patient's face during molding;
wherein the thermoformable seal is configured to be moldable against the patient's face while softened to conform to the patient's facial contours;
wherein the thermoformable seal retains at least part of a molded customized shape after cooling below the temperature range;
wherein the thermoformable seal is substantially rigid at temperatures below the softening temperature range;
and wherein the rigid frame remains substantially rigid at temperatures below 100° C.

2. The patient interface as claimed in claim 1, wherein the thermoformable seal substantially returns toward the non-customized shape after being reheated above its softening temperature, has a hardness greater than 15 Shore D at temperatures between 10° C. and 30° C., and has a hardness below Shore A 10 when heated above its softening temperature.

3. The patient interface as claimed in claim 1, wherein at least a portion of the facial-contacting portion of the thermoformable seal is substantially free of silicone or soft elastomer material.

4. The patient interface as claimed in claim 1, wherein a substantial portion of the facial-contacting portion of the thermoformable seal is configured to form a sealing interface with the patient's face without curving inwardly toward the breathing chamber where the thermoformable seal contacts the patient's face.

5. The patient interface as claimed in claim 4, wherein the thermoformable seal further comprises a silicone layer positioned over at least a portion of the thermoformable seal.

6. The patient interface as claimed in claim 1, wherein the thermoformable seal extends beyond the outer perimeter of the rigid frame in at least a cheek region or a nasal bridge region and forms a seal with a high aspect ratio where the thermoformable seal contacts the patient's face, the high aspect ratio being between 6.67 and 20, the high aspect ratio defined as a ratio of seal extension beyond the rigid frame to seal thickness.

7. The patient interface as claimed in claim 1, wherein the facial-contacting portion of the thermoformable seal is configured to distribute mask retention forces over an enlarged facial contact area to reduce localized facial pressure.

8. A patient interface comprising:
a rigid frame defining a breathing chamber configured to receive pressurized respiratory gas; and
a thermoformable seal coupled to the frame;
wherein the thermoformable seal comprises cross-linked polycaprolactone directly connected to the rigid frame and initially provided in a non-customized shape;
wherein the thermoformable seal softens when heated to a temperature of about 50-70° C.;
wherein the cross-linked polycaprolactone is configured to retain at least part of the non-customized shape of the thermoformable seal when heated above the softening temperature;
wherein the cross-linked polycaprolactone is configured for direct contact with the patient's face during molding;
wherein the thermoformable seal is configured to be moldable against the patient's face while softened to conform to the patient's facial contours;
wherein the thermoformable seal retains at least part of a molded customized shape after cooling below the temperature range;
wherein the thermoformable seal is substantially rigid at temperatures below the softening temperature range;
wherein the rigid frame includes a stabilizing mechanism positioned relative to the thermoformable seal to substantially constrain deformation of the thermoformable seal during molding in cheek regions or upper lip regions;
wherein the stabilizing mechanism is configured to provide a positive reference point for location of the mask assembly relative to the patient's cheek or upper lip to stabilize the mask assembly in a correct posterior and anterior location;
wherein adjacent portions of the thermoformable seal are permitted to deform more than the constrained regions during molding;
and wherein the rigid frame remains substantially rigid at temperatures below 100° C.

9. The patient interface as claimed in claim 8, wherein the thermoformable seal substantially returns toward the non-customized shape after being reheated above its softening temperature, has a hardness greater than 15 Shore D at temperatures between 10° C. and 30° C., and has a hardness below Shore A 10 when heated above its softening temperature.

10. The patient interface as claimed in claim 8, wherein at least a portion of the facial-contacting portion of the thermoformable seal is substantially free of silicone or soft elastomer material.

11. The patient interface as claimed in claim 8, wherein a substantial portion of the facial-contacting portion of the thermoformable seal is configured to form a sealing interface with the patient's face without curving inwardly toward the breathing chamber where the thermoformable seal contacts the patient's face.

12. The patient interface as claimed in claim 11, wherein the thermoformable seal further comprises a silicone layer positioned over at least a portion of the thermoformable seal.

13. The patient interface as claimed in claim 8, wherein the thermoformable seal extends beyond the outer perimeter of the rigid frame in at least a cheek region or a nasal bridge region and forms a seal with a high aspect ratio where the thermoformable seal contacts the patient's face, the high aspect ratio being between 6.67 and 20, the high aspect ratio defined as a ratio of seal extension beyond the rigid frame to seal thickness.

14. The patient interface as claimed in claim 8, wherein the thermoformable seal is permanently connected to, injection molded to, bonded to, or glued to the rigid frame.

15. A patient interface comprising:
a rigid frame defining a breathing chamber configured to receive pressurized respiratory gas; and
a thermoformable seal coupled to the frame and configured to form a sealing interface with a user during use;
wherein the thermoformable seal comprises cross-linked polycaprolactone, the cross-linked polycaprolactone being connected to the rigid frame and initially provided in a non-customized shape;
wherein a substantial portion of the facial-contacting portion of the thermoformable seal is positioned beyond the outer perimeter of the portion of the rigid frame forming the breathing chamber, in a cheek region or a nasal bridge region, as measured in the coronal plane;
wherein the thermoformable seal softens when heated to a temperature of about 50-70° C.;
wherein the thermoformable seal is configured to be moldable against the patient's face while softened to conform to the patient's facial contours;
wherein the thermoformable seal retains at least part of a molded customized shape after cooling below the temperature range;
wherein the thermoformable seal is substantially rigid at temperatures below the softening temperature range;
wherein an outer perimeter of the thermoformable seal where the thermoformable seal contacts the patient's face forms a projected seal area calculated in the coronal plane, and an outer perimeter of the rigid frame where the rigid frame forms the breathing chamber forms a projected frame area calculated in the coronal plane, the projected seal area being at least 1.5 times greater than the projected frame area;
and wherein the rigid frame remains substantially rigid at temperatures below 100° C.

16. The patient interface as claimed in claim 15, wherein the thermoformable seal substantially returns toward the non-customized shape after being reheated above its softening temperature, has a hardness greater than 15 Shore D at temperatures between 10° C. and 30° C., and has a hardness below Shore A 10 when heated above its softening temperature.

17. The patient interface as claimed in claim 15, wherein the projected seal area is between 1.5 and 2.5 times greater than the projected frame area.

18. The patient interface as claimed in claim 15, wherein a substantial portion of the facial-contacting portion of the thermoformable seal is configured to form a sealing interface with the patient's face without curving inwardly toward the breathing chamber where the thermoformable seal contacts the patient's face.

19. The patient interface as claimed in claim 18, wherein the thermoformable seal further comprises a silicone layer positioned over at least a portion of the thermoformable seal.

20. The patient interface as claimed in claim 15, wherein the thermoformable seal extends beyond the outer perimeter of the rigid frame in at least a cheek region or a nasal bridge region and forms a seal with a high aspect ratio where the thermoformable seal contacts the patient's face, the high aspect ratio being between 6.67 and 20, the high aspect ratio defined as a ratio of seal extension beyond the rigid frame to seal thickness.
